(19) United States
(12) Patent Application Publication
(10) Pub. No.: US 2024/0349992 A1
(43) Pub. Date: Oct. 24, 2024

(54) IMAGING DEVICE WITH ELONGATE ARM AND PIVOTABLE CAMERA

(71) Applicant: MOMENTIS SURGICAL LTD., Or Yehuda (IL)

(72) Inventors: Dvir COHEN, Ramot-Menashe (IL); Yaron LEVINSON, Tel-Aviv (IL); Idan ROTEM, Tel Aviv (IL); Yiftah NETA, Gilon (IL)

(21) Appl. No.: 18/685,797

(22) PCT Filed: Aug. 24, 2021
(86) PCT No.: PCT/IB2021/057753

§ 371 Date: Feb. 22, 2024

Related U.S. Application Data
(63) Continuation-in-part of application No. 17/109,354, filed on Dec. 2, 2020, now abandoned.
Application No. 17/109,354 is a continuation-in-part of application No. 16/776,548, filed on Jan. 30, 2020, now abandoned.
(60) Provisional application No. 62/798,508, filed on Jan. 30, 2019.

(51) Int. Cl. A61B 1/00; A61B 1/008; A61B 1/05; H04N 23/661; H04N 23/695

(52) U.S. Cl. CPC A61B 1/00183; A61B 1/00006; A61B 1/008; A61B 1/051; H04N 23/661; H04N 23/695

(57) ABSTRACT

An imaging device for capturing an image within a human body comprises an elongate mechanical arm and an imaging assembly coupled to the arm at a distal arm-location. The imaging assembly comprises a camera, a pivot member defining a pivot axis and engaged with the camera such that the camera is pivotable about the pivot axis throughout a pivot range, and an actuation member configured to pivot the camera in response to a remote user input. The pivot range can include a point at which an orientation of the camera is rotated at least 90° from an orientation of the elongate arm at the distal arm-location. The wherein the pivot range can include at least a range of 45° in at least a plane parallel to a longitudinal centerline of the elongate arm at the distal arm-location.

### CROSS-REFERENCE TO RELATED APPLICATIONS

This patent application is a continuation-in-part of U.S. Non-Provisional patent application Ser. No. 17/109,354, filed on Dec. 2, 2020, which is incorporated herein by reference in its entirety. U.S. Non-Provisional patent application Ser. No. 17/109,354 is a continuation-in-part of U.S. patent application Ser. No. 16/776,548, filed on Jan. 30, 2020, which is incorporated herein by reference in its entirety. U.S. patent application Ser. No. 16/776,548 claims the benefit of priority of U.S. Provisional Patent Application No. 62/798,508 filed on Jan. 30, 2019, which is incorporated herein by reference in its entirety.

### FIELD OF THE INVENTION

The present invention relates to pivotable cameras for use at the distal ends of elongate arms and in particularly to remotely pivotable cameras at the distal ends of remotely manipulable elongate arms.

### BACKGROUND

Cameras and similar imaging devices for use at the distal ends of manipulable arms include some endoscopes and non-medical instruments in which the camera angle is fixed relative to an orientation of the end of the arm, and camera orientation is dependent upon manipulation of the arm. Conversely, manually orientable cameras are available that are orientable independently of arm orientation but that do not offer remote orientation of the camera.

### SUMMARY OF THE INVENTION

According to embodiments disclosed herein, an imaging device for capturing an image within a human body comprises: (a) an elongate mechanical arm; and (b) an imaging assembly coupled to the arm at a distal arm-location, the imaging assembly comprising: (i) a camera, (ii) a pivot member defining a pivot axis orthogonal to a longitudinal centerline of the elongate arm at the distal arm-location the pivot member engaged with the camera such that the camera is pivotable about the pivot axis throughout a pivot range, and (iii) an actuation member configured to pivot the camera in response to a remote user input. The pivot range includes a point at which an orientation of the camera is rotated at least 90° from an orientation of the elongate arm at the distal arm-location. In some embodiments, pivoting the camera can include pivoting the camera to an orientation that is rotated at least 135° from an orientation of the elongate arm at the distal arm-location. In some embodiments, the pivot range can include at least a range of 45°. In some embodiments, the pivot range can be independent of a distal-arm-location orientation.

In some embodiments, the elongate mechanical arm can be remotely user-manipulable. In some embodiments, the elongate mechanical arm can be configured for flexing. In some embodiments, the elongate mechanical arm can comprise a plurality of arm segments connected serially by arm joints having respective degrees of freedom and configured to flex and rotate in response to remote user inputs.

In some embodiments, the pivot range includes at least a range of 45° or at least 90° at least in a plane parallel to a longitudinal centerline of the elongate arm at the distal arm-location. In some embodiments, the pivot member can be transversely connected to the distal portion of the elongate arm.

In some embodiments, the pivot range can be independent of a distal-arm-location orientation.

In some embodiments, the actuation member can be powered by a local power source at the distal arm-location. In some embodiments, the actuation member can be powered by a remote power source.

In some embodiments, the actuation member can include a micro-electromechanical system.

In some embodiments, the remote user input can be received locally at the distal arm-location by the actuation member.

In some embodiments, the imaging assembly can additionally include electronic communications circuitry in data communication with the actuation member and configured to receive a remote user input.

In some embodiments, the remote user input can be received remotely by the actuation member.

In some embodiments, the remote user input can be received in a proximal portion of the elongated arm.

In some embodiments, the pivot range can include at least a range of 90° in at least the plane parallel to the longitudinal centerline of the elongate arm at the distal arm-location. In some embodiments, the pivot range can include at least a range of 135° in at least the plane parallel to the longitudinal centerline of the elongate arm at the distal arm-location.

In some embodiments, the camera can be pivotable about multiple pivot axes. In some embodiments, the pivot range can include a range of 360° in at least one plane.

In some embodiments, the pivot member can be part of a 3-axis gimbal arrangement disposed at the distal-arm location.

In some embodiments, the pivot range can include a range defining a pivoting of the camera about the longitudinal centerline of the elongate arm at the distal arm-location.

A method is disclosed, according to embodiments, for capturing an image. The method comprises: (a) providing an imaging device comprising (i) a remotely-manipulable elongate mechanical arm, and (ii) an imaging assembly coupled to the arm at a distal arm-location, the imaging assembly comprising a camera, a pivot member defining a pivot axis and engaged with the camera, and an actuation member configured to pivot the camera about the pivot axis; (b) receiving, by the actuation member, a remote user input; (c) in response to the remote user input, pivoting the camera, by the actuation member, in a pivot range that includes an orientation at which the camera is rotated at least 90° from the longitudinal centerline of the elongate arm at the distal arm-location; and (d) subsequent to the pivoting, capturing an image at the imaging location.

In some embodiments, the navigating can include remotely manipulating the elongate mechanical arm.

In some embodiments, the navigating can include flexing the elongate mechanical arm.

In some embodiments, the elongate mechanical arm can be a remotely-manipulable arm comprising a plurality of arm segments connected serially by arm joints having respective degrees of freedom and configured to flex and rotate in response to remote user inputs.

In some embodiments, the pivot axis can be oriented transversely to the longitudinal centerline of the distal portion of the elongate arm. In some embodiments, the pivot member can be transversely connected to the distal portion of the elongate arm.

In some embodiments, the pivot range can be independent of a distal-arm-location orientation.

In some embodiments, pivoting the camera by the actuation member can include receiving electrical power from a local power source at the distal arm-location. In some embodiments, pivoting the camera by the actuation member can include receiving electrical power from a remote power source.

In some embodiments, the actuation member can include a micro-electromechanical system.

In some embodiments, receiving the remote user input by the actuation member can include receiving the remote user input locally at the distal arm-location.

In some embodiments, the imaging assembly can additionally include electronic communications circuitry in data communication with the actuation member, and receiving the remote user input includes receiving the remote user input by the electronic communications circuitry.

In some embodiments, receiving the remote user input by the actuation member can include receiving the remote user input remotely.

In some embodiments, receiving the remote user input by the actuation member can include receiving the remote user input in a proximal portion of the elongated arm.

In some embodiments, pivoting the camera can include pivoting the camera through a pivot range of at least 90°. In some embodiments, pivoting the camera can include pivoting the camera through a pivot range of at least 90°.

In some embodiments, pivoting the camera can include pivoting the camera to an orientation that is rotated at least 90° from an orientation of the elongate arm at the distal arm-location. In some embodiments, pivoting the camera can include pivoting the camera to an orientation that is rotated at least 135° from an orientation of the elongate arm at the distal arm-location.

In some embodiments, pivoting the camera can include pivoting the camera about multiple pivot axes. In some embodiments, the pivot range can include a range of 360° in at least one plane.

In some embodiments, pivoting the camera can include pivoting the camera in a 3-axis gimbal arrangement disposed at the distal-arm location.

In some embodiments, pivoting the camera can include pivoting the camera about the longitudinal centerline of the elongate arm at the distal arm-location.

According to embodiments disclosed herein, an imaging device, e.g., an imaging device for capturing an image within a human body, comprises: (a) an elongate mechanical arm; and (b) an imaging assembly coupled to the arm at a distal arm-location. The imaging assembly comprises: (i) a camera, (ii) a pivot member defining a pivot axis orthogonal to a longitudinal centerline of the elongate arm at the distal arm-location, the pivot member engaged with the camera such that the camera is pivotable about the pivot axis throughout a pivot range, and (iii) an actuation member configured to pivot the camera in response to a remote user input. The pivot range includes at least a range of 45°. In some embodiments, the pivot range can include at least a range of 90°. In some embodiments, the pivot range can include at least a range of 135°.

A method is disclosed according to embodiments, for capturing an image within a human body. The method comprises: (a) navigating, to an imaging location within the body, an imaging device comprising (i) an elongate arm and (ii) an imaging assembly coupled to the arm at a distal arm-location, the imaging assembly comprising a camera, a pivot member defining a pivot axis orthogonal to a longitudinal centerline of the elongate arm at the distal arm-location and engaged with the camera, and an actuation member configured to pivot the camera about the pivot axis; (b) receiving, by the actuation member, a remote user input; (c) in response to the remote user input, pivoting the camera by the actuation member, through a pivot range of at least 45°; and (d) subsequent to the pivoting, capturing an image at the imaging location.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic perspective view of an imaging device according to embodiments of the present invention.

FIGS. 2A and 2B are schematic drawings of an elongate arm and imaging assembly according to embodiments of the present invention.

FIGS. 3A, 3B and 3C show the elongate arm and imaging assembly of FIGS. 2A-B along with various pivot ranges of a camera, according to embodiments of the present invention.

FIGS. 4A and 4B show the elongate arm and imaging assembly of FIGS. 2A-B along with various relative-to-arm pivot angles of a camera, according to embodiments of the present invention.

FIGS. 5A, 5B and 5C are schematic illustrations of an elongate arm and an imaging assembly comprising a pivot axis, according to embodiments of the present invention.

FIGS. 6A, 6B and 6C are schematic illustrations of a flexible elongate arm and an imaging assembly comprising a pivot axis, according to embodiments of the present invention.

FIGS. 7A, 7B, 7C, 7D and 7E are schematic illustrations of a flexible elongate arm and an imaging assembly comprising a 3-axis gimbal arrangement, according to embodiments of the present invention.

FIG. 8 is a schematic illustration of an imaging assembly having remote connections for actuation, power and communications, according to embodiments of the present invention.

FIG. 9A shows an elongate arm and the imaging assembly of FIG. 8, according to embodiments of the present invention.

FIG. 9B shows a proximal detail of the elongate arm of FIG. 9A including remote actuation, power, and communications arrangements, according to embodiments of the present invention.

FIG. 10 is a schematic illustration of an imaging assembly having local for actuation, power, and communications arrangements, according to embodiments of the present invention.

FIG. 11 is a schematic illustration of a housing unit for an elongate arm including remote actuation, power, and communications arrangements within the housing unit, according to embodiments of the present invention.

FIGS. 12 and 13 show flowcharts of method steps for capturing images, according to embodiments of the present invention.

FIGS. 14A-16 schematically illustrate structures of imaging devices according to embodiments of the present invention.

FIG. 17 is a flowchart of general use of a medical robotic surgical system, according to some embodiments.

FIG. 18A is a simplified schematic drawing of a surgical system 200, according to some embodiments.

FIG. 18B is an example of an operating room setting, according to some embodiments.

FIG. 18C is a simplified schematic drawing of one or more surgical arms held by a support (e.g., a fixation arm), according to some embodiments.

FIG. 18D is a simplified schematic drawing of a surgical mechanical arm controlled by an input arm, according to some embodiments.

FIG. 19 is a flow chart of a detailed use flow of a medical robotic surgical system, according to some embodiments;

FIGS. 20A-C are a flowchart of a method for dual-control of surgical arms (FIG. 4A) and schematic drawings of a control console comprising dual control means (FIGS. 4A-B), according to some embodiments;

FIG. 21 is a flowchart of a method of using haptic handles to control one or more surgical arms, according to some embodiments;

FIGS. 22A-C illustrate stages and tools used during an emergency release of the surgical arms, according to some embodiments;

FIGS. 23A-B schematically illustrates an exemplary surgical setup for transvaginal access, according to some embodiments;

FIGS. 24A-B are drawings of a fixation arm (24A) configured to engage a mounting block (24B), according to some embodiments

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

Embodiments disclosed herein relate to imaging devices comprising elongated arms and imaging assemblies. Imaging assemblies, according to embodiments, include one or more cameras pivotably installed at the distal ends of the elongated arms, a pivot member around which the camera is pivotable throughout a pivot range, and an actuation member configured to pivot the camera in response to a remote user input. The term ‘camera’ is used broadly herein to refer to any imaging device for capturing, transmitting and/or storing a still or video image.

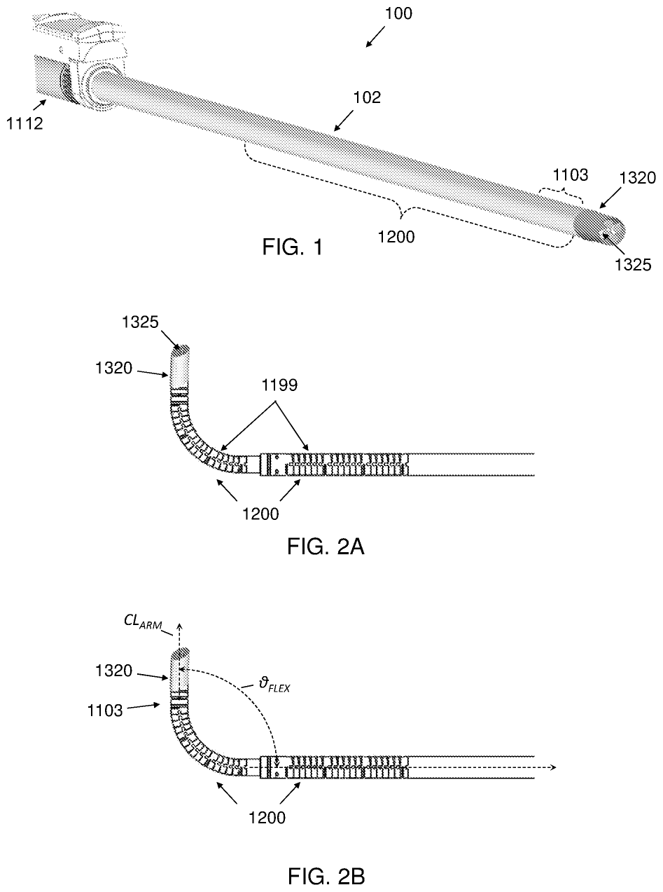

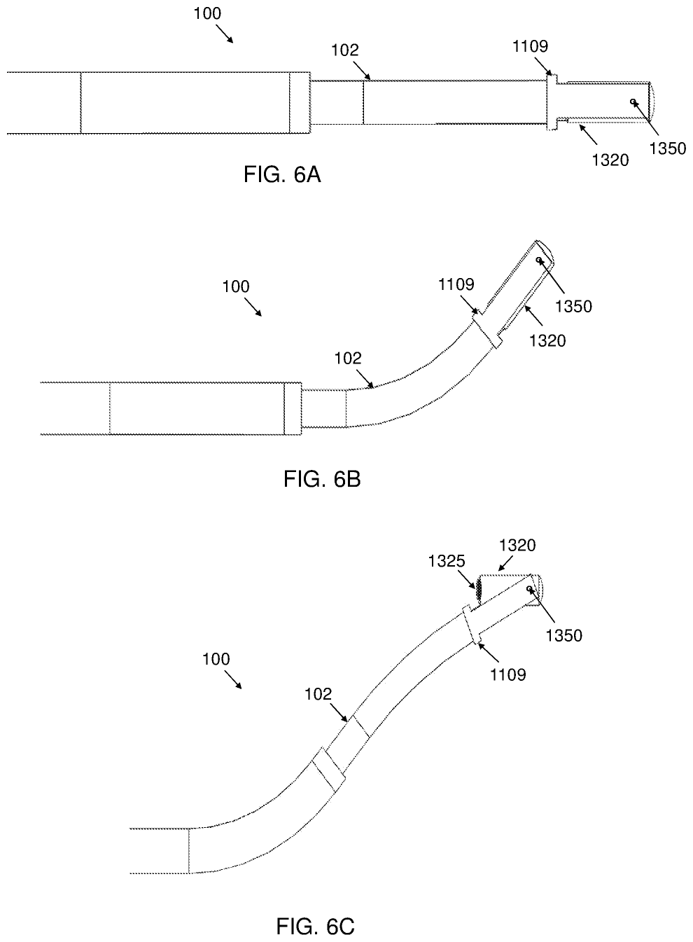

Referring now to the figures and in particular to FIG. 1, an imaging device 100 includes an elongated arm 102 and an imaging assembly 1320 coupled, e.g., attached, installed or detachedly attached, to a distal end of the arm 102. The imaging assembly 1320 includes at least one camera 1325; while the camera is shown as facing distally, this is not necessarily so and in other examples, such as that shown in FIGS. 6A, 6B and 6C, the camera can ‘default’ to facing backwards, i.e., proximally. The terms ‘distal’ and ‘proximal’ as used herein and in the appended claims are used to indicate, respectively, at or towards the end of the arm to which the camera is coupled, and at or towards the other end of the arm, which generally is the end held by a user or supported by a housing, etc. A non-limiting example of the distal portion 1103 of the arm 102 is illustrated in FIG. 1. The term ‘longitudinal’ as used herein is the direction connecting proximal and distal ends.

At least a portion 1200 of the arm 102 can be flexible. In some specific implementations, the flexible portion includes articulated sections and joints. FIGS. 2A and 2B show an arm design in which the flexible portion 1200 comprises a series of ‘stacked links’ 1199 that enable flexibility for the outer contour/surface of the arm 102. As indicated in FIG. 2B, the flexibility and/or articulation of the arm can allow the distal end 1103 of the arm 102 to be flexed. In the non-limiting example of FIG. 2B, the distal end 1103 of the arm 102, specifically a longitudinal centerline CL_{ARM} of the arm 102 at its distal end 1103, is oriented at a flex angle θ_{FLEX} from the non-flexed portion. Depending on arm design, the flex angle θ_{FLEX} can be at least 90°, or at least 120°, or at least 150°, or at least 180°, or at least 210°.

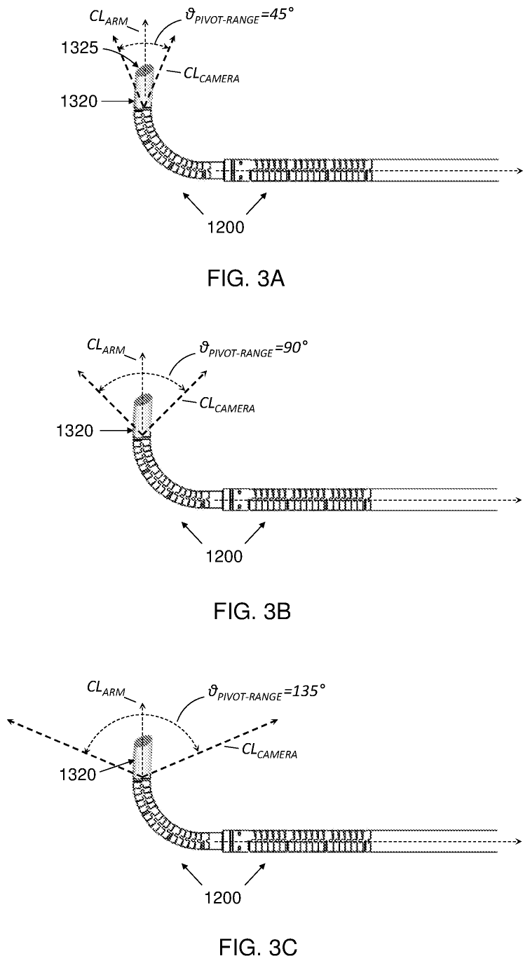

In embodiments, an imaging assembly 1320 coupled to the distal end 1103 of an elongate arm 102 can be arranged to pivot so that the camera 1325 has a pivot range independent of the orientation of the arm 102 i.e., independent of the orientation of its distal end 1103 or independent of its flex angle θ_{FLEX}. In the schematic illustrations of FIGS. 3A, 3B and 3C, respective pivot ranges θ_{PIVOT-RANGE} of 45°, 90° and 135° are illustrated. In each of the figures, the pivot range indicates a range of rotation of a longitudinal centerline CL_{CAMERA} of the camera 1325. The pivot range θ_{PIVOT-RANGE} is not relative to the arm or to the longitudinal centerline CL_{ARM} of the arm 102 at its distal end 1103. In some designs, the camera 1325 is pivotable as part of the camera assembly 1320, i.e., the camera assembly 1320 is pivotable as a unit. In some other designs the camera 1325 is pivotable independently of the camera assembly 1320. In yet other designs, the camera 1325 is pivotable together with one or more other components of the camera assembly 1320 but the entire camera assembly 1320 does not pivot as a unit. This disclosure does not distinguish further between these design options, and all such design options are within the scope of the present invention. Thus, an expression such as ‘pivoting the camera 1325’ may or may not include the pivoting of the camera assembly 1320 or any other part of the camera assembly 1320.

A camera 1325, e.g., defined by a camera centerline CL_{CAMERA}, can pivot in multiple planes; according to embodiments, and as illustrated in FIGS. 3A-C, at least one of the planes through which it pivots is parallel to the arm centerline CL_{ARM}. While the respective pivot ranges θ_{PIVOT-RANGE} are shown for convenience in FIGS. 3A-C as being centered around the arm centerline CL_{ARM}, this is not necessarily so. As an illustrative example (not shown), a camera may have a pivot range θ_{PIVOT-RANGE} equal to at least 45° where the pivot range is entirely to one side of the arm centerline CL_{ARM}.

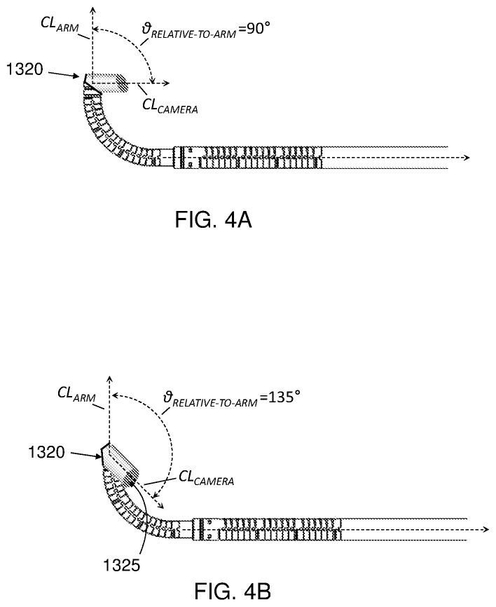

In contrast to FIGS. 3A-C, FIGS. 4A and 4B illustrate a pivot range θ_{RELATIVE-TO-ARM} that is defined relative to the arm centerline CL_{ARM}. FIG. 4A shows a camera 1325—together with the entire imaging assembly 1320—rotated at the extreme point of a pivot range θ_{RELATIVE-TO-ARM} of 90°. FIG. 4A shows the camera 1325 rotated at the extreme point of a pivot range θ_{RELATIVE-TO-ARM} of 135°. Thus, while the pivoting of the camera 1325 is independent of the orientation of the arm 102, it is possible to define pivot ranges θ_{PIVOT-RANGE} measured independently, i.e., the arc from one extreme to the other, or pivot ranges θ_{RELATIVE-TO-ARM} measured from the arm centerline CL_{ARM} to its extreme point away from the arm centerline CL_{ARM}. Thus, the 90° pivot range θ_{RELATIVE-TO-ARM} of FIG. 4A and the θ_{PIVOT-RANGE} of 90° of FIG. 3B are the same range, if the latter extends from a first extreme point at the arm centerline CL_{ARM} to a 2nd extreme point 90° away.

An aspect of some embodiments relates to an imager (e.g., a camera) for use during surgery, the imager configured for retroflection inside the body while potentially avoiding blockage of the field of view by adjacent organs. In some embodiments, the camera itself is configured to be retroflected, for example positioned at an angle with respect to the arm from which it extends; additionally or alternatively, the arm from which the camera extends is flexible and can be bent to position the camera to face backwards (e.g. opposite the direction of advancement of the arm into the body).

Some embodiments comprise a camera mounted on a head configured for pivoting with respect to a arm from which it extends. In some embodiments, the arm comprises a distal rigid extension which defines a recess in which the head is received, and from which it can be pivoted outwardly, for example to position the camera at an angle relative to the rigid extension (e.g. an angle of between 5-135 degrees). Optionally, when the head is received within the recess, it is flush with the rigid extension.

In some embodiments, a gimbal support is provided, and one or more cameras are mounted onto the gimbal support. Optionally, the gimbal support is configured for rotating with respect to an arm from which it extends, to position the one or more cameras at a selected angle and/or orientation.

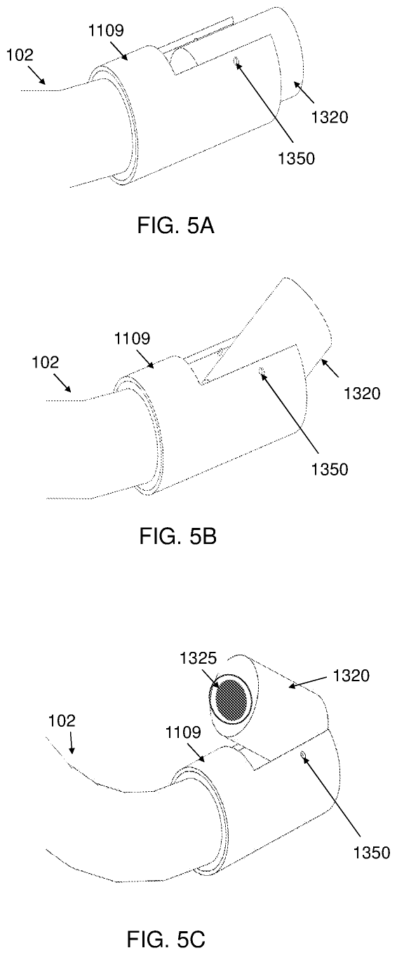

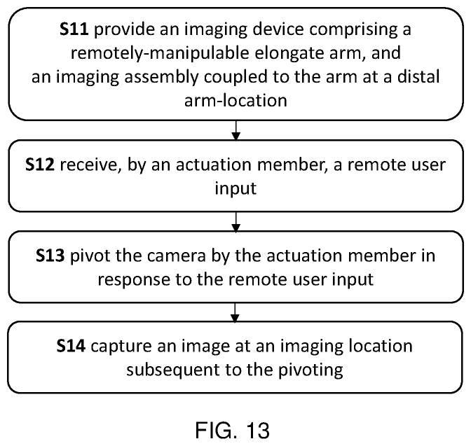

FIGS. 5A, 5B and 5C show an imaging assembly 1320 onto which the camera 1325 (not shown in FIGS. 5A-B) is mounted, according to some embodiments. The imaging assembly 1320 is configured for pivoting with respect to an arm extension 1109 from which it extends. The arm extension 1109 is an optional connector used for coupling the imaging assembly 1320 to the arm 102. In some embodiments, as shown for example in FIG. 5A, the imaging assembly 1320 is at least partially received within the arm extension 1109, optionally aligned longitudinally with the arm extension 1109. In FIG. 13B, the imaging assembly 1320 is slightly pivoted, for example so as to position the camera 1325 at a 45° angle with respect to the arm extension 1109. In FIG. 5C, the imaging assembly 1320 is pivoted to position the camera 1325 at an angle of 135° with respect to the orientation of the arm extension 1109, thereby obtaining a ‘backwards-looking view when imaging. In embodiments, the pivoting is about a pivot member 1350 which defines the pivot axis around which the imaging assembly 1320 and the camera 1325 are pivotable.

FIGS. 6A, 6B and 6C illustrate another camera 1325 mounted onto a pivoting imaging assembly 1320, according to some embodiments. The imaging assembly 1320 is received within a recess defined by the arm extension 1109 in a manner in which the imaging assembly 1320 can be moved from a position in which it is flush with the arm extension 1109 to a position in the imaging assembly 1320 pivots away from the arm extension 1109 as in, for example, the orientation of FIG. 6B). This positions the camera 1325 in a ‘backwards-looking’ position in which the camera 1325 is positioned for capturing a view opposite the advancing direction of the arm 102. The arm 102 is shown in FIGS. 6B and 6C as being flexed into various orientations. In embodiments, pivoting of the imaging assembly 1320 and of the camera 1325 is about a pivot member 1350 located, in this example, at a distal end of the recess defined by the arm extension 1109.

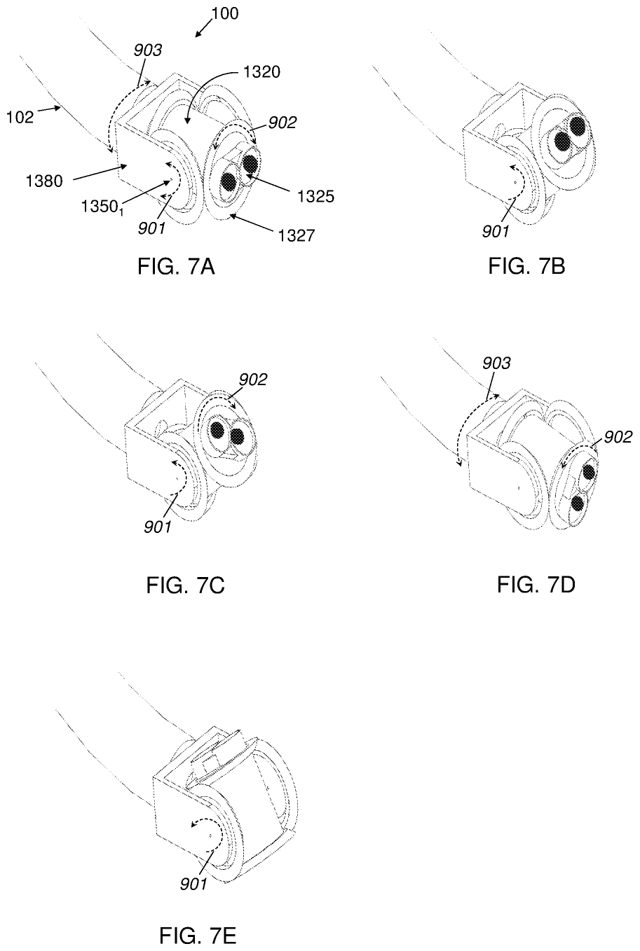

FIGS. 7A, 7B, 7C, 7D and 7E illustrate an imaging assembly construction including gimbal support for pivoting around multiple pivot members 1350 defining respective pivot axes in multiple pivot planes, according to some embodiments. One or more cameras 1325 are positioned on an imaging assembly 1320 mounted in a 3-axis gimbal arrangement 1380. In embodiments, positioning of the one or more cameras 1325 to capture a desired view is performed by one or more of:
pivoting the imaging assembly 1320 around the first pivot member 13501 relative to the elongate arm 102 as indicated by arrow 901 in FIGS. 7A, 7B, 7C and 7D, including the backwards-facing orientation of FIG. 7E;
rotating a camera mounting 1327 supporting the camera(s) 1325, relative to the main body of the imaging assembly 1320 as indicated by arrow 902 in FIGS. 7A, 7C and 7D; and
rotating the imaging assembly 1320 relative to the arm 102 as indicated by arrow 903 in FIGS. 7A and 7D. The camera orientation shown in FIG. 7D can be achieved by rotations according to either or both of arrows 902 and 903.

In some embodiments, an arrangement of two or more cameras 1325 is provided in a camera assembly 1320 as shown in FIGS. 7A-D. Optionally, the cameras 1325 are positioned to face a similar direction. Alternatively, different cameras 1325 face different directions. A potential advantage of using two or more cameras may include improving vision, for example, for obtaining 3-dimensional vision.

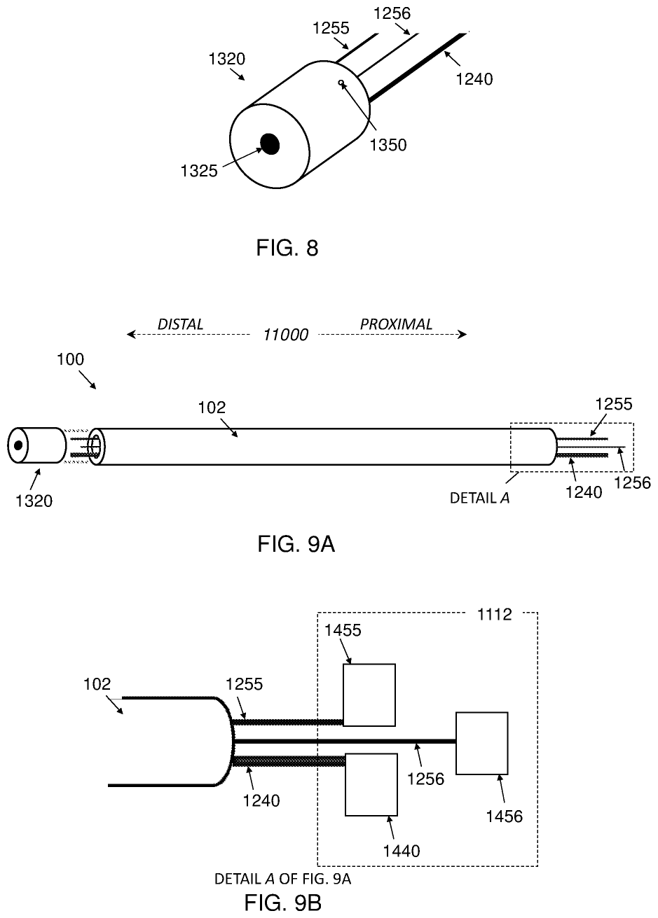

We now refer to FIGS. 8, 9A, 9B and 10.

In embodiments, a camera assembly 1320 comprises an actuation member configured to pivot a camera 1325 in response to a remote user input.

In a non-limiting example shown in FIG. 8, an actuation member includes an elongate actuation cable 1240 extending at least from a proximal portion of an elongate arm to the camera assembly 1320 at the distal end 1103 of the arm 102. An actuation cable 1240 can be coupled to the camera assembly 1320 such that longitudinal manipulation, e.g., pushing or pulling, of the actuation cable 1240 causes the camera assembly 1320 to pivot about its pivot member 1350. In the example of FIG. 8, manipulation of the actuation cable 1240 is in response to a remote user input received by the actuation member (e.g., actuation cable 1240) remotely from the camera assembly 1320. For example, a user input can include direct or indirect manipulation of a proximal portion of the actuation cable 1240.

In some embodiments, the remote user input is received by the actuation member (e.g., actuation cable 1240) in a proximal portion of the elongate arm 102. In other embodiments, the actuation cable 1240 extends proximally from a proximal end of the elongate arm 102, and the remote user input can be received outside of the elongate arm 102. In the examples of FIGS. 9A and 9B, the actuation cable 1240 extends proximally from the proximal end of the elongate arm 102, where the proximal and distal directions are indicated in FIG. 9A by arrow 11000. In the example of FIG. 9B, the actuation cable 1240 is in at least indirect communication with an actuation-control device 1440 which can be used to transfer a remote user input to the actuation cable 1240. Suitable examples of actuation control-devices 1440 include electronic and/or mechanical controls causing longitudinal manipulation of the actuation cable 1240 in response to a user input entered through an input device. In another example (not shown), the actuation control-device 1440 is in communication with a proximal portion of the actuation cable 1240 disposed within a proximal portion of the elongate arm 102.

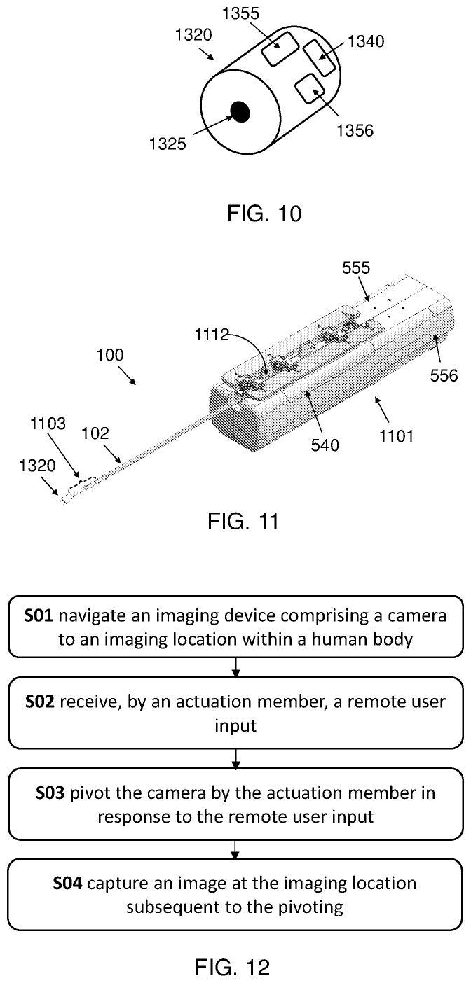

In a non-limiting example shown in FIG. 10, an actuation member includes a locally-disposed actuation member 1340 arranged to pivot the camera 1325 in response to a remote user input A suitable example of a locally-disposed actuation member 1340 is a microelectromechanical system (MEMS). The MEMS can be arranged to be in at least indirect contact with a pivot member 1350 so as to cause the camera 1325 to pivot upon receipt of a remote user input. Another suitable example of a locally-disposed actuation member 1340 is miniature snap-action switch such as the ‘Micro Switch TM’ products known in the electrical and mechanical industries.

A locally-disposed actuation member 1340 such as a MEMS or micro switch can be powered by a local power source or by a remote power source. An example of a local power source is local power source 1355, shown in FIG. 10, disposed on or in electrical contact with the camera assembly 1320 and the locally-disposed actuation member 1340. An example of a remote power source is remote power source 4155, shown in FIG. 9B, located in or at a proximal end of the elongate arm 102 and in electrical communication with the camera assembly, e.g., with the locally-disposed actuation member 1340, via electrical cable 1255 shown in FIGS. 8, 9A and 9B. In embodiments, the electrical cable 1255 is provided for powering the camera 325 from a remote power source, e.g., remote power source 1455, regardless of whether the electrical cable 1255 is additionally provided for powering a local locally-disposed actuation member 1340.

A locally-disposed actuation member 3140 can receive a remote user input, e.g., a remotely-generated user input, through electronic communications circuitry. A first example of electronic communications circuitry includes, and not exhaustively, a locally-disposed wireless communications arrangement 1356, shown in FIG. 10, disposed on or in at least indirect contact with the camera assembly 1320 and the locally-disposed actuation member 1340. A second example of electronic communications circuitry includes a communications cable 1256 shown in FIGS. 8, 9A and 9B.

In an exemplary design, a remote user input causing the locally-disposed actuation member 1340 to pivot the camera is received locally, i.e., at the camera assembly 1320, by the locally-disposed wireless communications arrangement 1356. In another exemplary design (not illustrated), a remote user input causing the locally-disposed actuation member 1340 to pivot the camera is received remotely, for example at a communications unit 1456, shown in FIG. 9B, located in or at a proximal end of the elongate arm 102 and in data communication with the camera assembly 1320, e.g., with the locally-disposed actuation member 1340, via communications cable 1256. In embodiments, the electronic communications circuitry is provided for communicating with the camera 1325, e.g., for controlling imaging and transmitting (from the camera 1325) captured images, regardless of whether the electronic communications circuitry is additionally provided for transmitting a remote user input for causing an actuation member to pivot the camera 1325.

FIG. 11 includes a schematic drawing of a motor-control unit 1101 for an imaging device 100 suitable for use in capturing an image at an imaging location within a human body, the imaging device 100 comprising an elongate arm 102 and an imaging assembly 1320 coupled to the distal end 1103 of the elongate arm 102. The imaging device 100 includes a proximal arm extension 1112 which interfaces with the motor control unit 1101. According to embodiments, the motor unit can have housed therewithin one or more of: a remote power source 555 for powering the actuation member, e.g., additionally or alternatively to remote power source 1455 of FIG. 9B; communications unit 556, e.g., additionally or alternatively to remote communications unit 1456 of FIG. 9B; and a remote actuation control-device 540, e.g., additionally or alternatively to actuation control-device 1440 of FIG. 9B.

In embodiments, an actuation member can be entirely locally disposed or not. In embodiments, a power source for powering the actuation member can be local or remote. In embodiments, a remote user input can be received locally or remotely. Any valid, i.e., practicable, combination of these design options is possible and falls within the scope of the present invention. In any of the design options, ‘local’ refers to at or in proximity to the camera assembly 1320 and/or camera 1325, while ‘remote’ can refer to one or more of: (i) a proximal portion of the elongate arm 102, (ii) proximal from the elongate arm 102, and (iii) within the motor control unit 1101.

Referring now to FIG. 12, a method is disclosed for capturing an image within a human body. As illustrated by the flowchart in FIG. 12, the method comprises Steps S01, S02, S03, and S04, which are discussed in the following paragraphs.

Step S01 includes navigating an imaging device 100 to an imaging location within the body. The imaging device 100 comprises (i) an elongate arm 102 and (ii) an imaging assembly 1320 coupled to the arm 102 at a distal arm-location 1103, the imaging assembly 1320 comprising a camera 1325, a pivot member 1350 defining a pivot axis and engaged with the camera, and an actuation member, e.g., elongate actuation cable 1240 or locally-disposed actuation member 1340, configured to pivot the camera about the pivot axis. In some embodiments, the pivot member 1350 is transversely connected, at least indirectly, to the distal portion 1103 of the elongate arm 102. In some embodiments of the method, the pivot axis is oriented transversely to the longitudinal centerline CL_{ARM} of the distal portion 1103 of the elongate arm 102.

In some embodiments, the navigating includes remotely manipulating and/or flexing the elongate arm 102. ‘Remotely manipulating’ (or ‘remotely manipulable’ and the like) means manipulation such as extension, retraction, rotation and/or flexion of all or part of the arm 102, performed indirectly using mechanical and/or electronic control elements and an input device or other control unit external to the arm. In some embodiments, the elongate arm 102 is a remotely-manipulable arm comprising a plurality of arm segments 1199 connected serially by arm joints having respective degrees of freedom and configured to flex and rotate in response to remote user inputs

Step S02 includes receiving, by the actuation member, e.g., elongate actuation cable 1240 or locally-disposed actuation member 1340, a remote user input. In some embodiments, the imaging assembly 1320 additionally includes electronic communications circuitry in data communication with the actuation member, and receiving the remote user input includes receiving the remote user input by the electronic communications circuitry, e.g., local communications unit 1356, remote communications unit 1456 or remote communications 556. In some embodiments, receiving the remote user input by the actuation member includes receiving the remote user input remotely—for example, in a proximal portion of the elongated arm 102.

Step S03 includes pivoting the camera by the actuation member, e.g., elongate actuation cable 1240 or locally-disposed actuation member 1340, in response to the remote user input received in Step S02. In some embodiments, receiving the remote user input by the actuation member includes receiving the remote user input locally at the distal arm-location. The pivot is in a pivot range θ_{PIVOT-RANGE} of at least 45°, or alternatively at least 90°, or alternatively at least 135°, in at least a plane parallel to a longitudinal centerline CL_{ARM} of a distal portion 1103 of the elongate arm 102. In some embodiments, the pivot range θ_{PIVOT-RANGE} is independent of a distal-arm-location orientation. In some embodiments, pivoting the camera 1325 includes pivoting the camera 1325 to an orientation θ_{RELATIVE-TO-ARM} that is rotated at least 90°, or alternatively at least 135°, from an orientation of the elongate arm at the distal arm-location.

In some embodiments, pivoting the camera 1325 includes pivoting the camera 1325 about multiple pivot axes. In some embodiments, the pivot member 1350 is part of a 3-axis gimbal arrangement 1380 disposed at the distal-arm location, and pivoting the camera 1325 includes pivoting the camera 1325 in the 3-axis gimbal arrangement. Pivoting the camera 1325 can include pivoting the camera 1325 about the longitudinal centerline CL_{ARM} of a distal portion 1103 of the elongate arm 102.

In some embodiments, pivoting the camera 1325 by the actuation member, e.g., elongate actuation cable 1240 or locally-disposed actuation member 1340, includes receiving electrical power from a local power source 355 at the distal arm-location, and in other embodiments it includes receiving electrical power from a remote power source, e.g., remote power source 1455 or remote power source 555.

Step S04 includes capturing an image at the imaging location subsequent to the pivoting.

In some embodiments of the method, the actuation member includes a micro-electromechanical system and/or a micro switch.

Referring now to FIG. 13, a method is disclosed for capturing an image. As illustrated by the flowchart in FIG. 13, the method comprises Steps S11, S12, S13, and S14, which are discussed in the following paragraphs.

Step S11 includes providing an imaging device 100 comprising a remotely-manipulable elongate mechanical arm 102, and an imaging assembly 1320 coupled to the arm 102 at a distal arm-location, the imaging assembly 1320 comprising a camera 1325, a pivot member 350 defining a pivot axis and engaged with the camera 1325, and an actuation member e.g., elongate actuation cable 1240 or locally-disposed actuation member 1340, configured to pivot the camera about the pivot axis. In some embodiments, the pivot member 1350 is transversely connected, at least indirectly, to the distal portion 1103 of the elongate arm 102. In some embodiments of the method, the pivot axis is oriented transversely to the longitudinal centerline CL_{ARM} of the distal portion 1103 of the elongate arm 102.

In some embodiments, the navigating includes remotely manipulating and/or flexing the elongate arm 102. In some embodiments, the elongate arm 102 is a remotely-manipulable arm comprising a plurality of arm segments 1199 connected serially by arm joints having respective degrees of freedom and configured to flex and rotate in response to remote user inputs

Step S12 includes receiving, by the actuation member, e.g., elongate actuation cable 1240 or locally-disposed actuation member 1340, a remote user input. In some embodiments, the imaging assembly 1320 additionally includes electronic communications circuitry in data communication with the actuation member, and receiving the remote user input includes receiving the remote user input by the electronic communications circuitry, e.g., local communications unit 1356, remote communications unit 1456 or remote communications 1556. In some embodiments, receiving the remote user input by the actuation member includes receiving the remote user input remotely—for example, in a proximal portion of the elongated arm 102.

Step S13 includes pivoting the camera by the actuation member, e.g., elongate actuation cable 1240 or locally-disposed actuation member 1340, in response to the remote user input received in Step S12. In some embodiments, receiving the remote user input by the actuation member includes receiving the remote user input locally at the distal arm-location. The pivot is in a pivot range θ_{PIVOT-RANGE} of at least 45°, or alternatively at least 90°, or alternatively at least 135°, in at least a plane parallel to a longitudinal centerline CL_{ARM} of a distal portion 103 of the elongate arm 102. In some embodiments, the pivot range θ_{PIVOT-RANGE} is independent of a distal-arm-location orientation. In some embodiments, pivoting the camera 1325 includes pivoting the camera 1325 to an orientation θ_{RELATIVE-TO-ARM} that is rotated at least 90°, or alternatively at least 135°, from an orientation of the elongate arm at the distal arm-location.

In some embodiments, pivoting the camera 1325 includes pivoting the camera 1325 about multiple pivot axes. In some embodiments, the pivot member 3150 is part of a 3-axis gimbal arrangement 1380 disposed at the distal-arm location, and pivoting the camera 325 includes pivoting the camera 1325 in the 3-axis gimbal arrangement. Pivoting the camera 325 can include pivoting the camera 1325 about the longitudinal centerline CL_{ARM} of a distal portion 1103 of the elongate arm 102.

In some embodiments, pivoting the camera 1325 by the actuation member, e.g., elongate actuation cable 1240 or locally-disposed actuation member 1340, includes receiving electrical power from a local power source 1355 at the distal arm-location, and in other embodiments it includes receiving electrical power from a remote power source, e.g., remote power source 1455 or remote power source 555.

Step S14 includes capturing an image at an imaging location subsequent to the pivoting.

In some embodiments of the method, the actuation member includes a micro-electromechanical system and/or a micro switch.

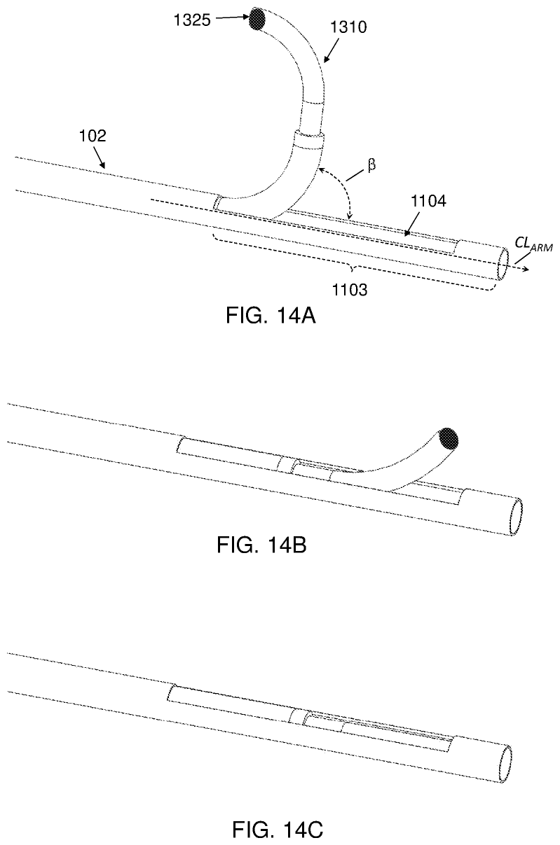

Reference is now made to FIGS. 14A, 14B, 14C, 15A, 15B and 16.

In some embodiments, the imager is configured for obtaining multiple visual reference points, for example by the user directing the camera at a plurality of directions, which may assist the user in determining a current anatomical position. In some embodiments, a camera is mounted onto a flexible extension configured to be advanced (and/or retracted) through a cannulated arm. In some embodiments, the arm is formed with a window, for example at a side wall of the arm, and the flexible extension can be pushed at least partially out the window to position the camera at a selected location and/or angle. Optionally, by manipulation of the flexible extension on which the camera is mounted, the camera can be turned (e.g. retroflected), for facing a direction opposite the direction of advancement of the arm. Optionally, a field of view obtained by the retroflected camera includes a working space in which the retroflected surgical arms operate.

In some embodiments, the flexible extension is positionable at an angle with respect to a longitudinal axis defined by the arm. In some embodiments, a far end of the flexible extension, onto which the camera is mounted, is coupled to a tensioning element, for example a wire cable, which extends to a distal opening of the arm. Optionally, upon pulling or releasing of the cable, a curvature of the flexible extension is changed. In an example, the extension is curved into an S-shaped curve, a C-shaped curve. A potential advantage of approximating the camera by manipulating a curvature of the flexible extension may include reducing a bending radius of the extension, thereby potentially facilitating turning or generally maneuvering the camera inside the patient body. In some embodiments, the arm through which the camera is advanced comprises a distal spacer for clearing a working space distally of the arm, for example by pushing away tissue. Optionally, the spacer comprises a ring or arc at its distal end. Optionally, the camera is advanceable through the ring or with respect to the arc. Potential advantages of a ring or arc configured at a distal end of the spacer may include: being aligned with a circumference of the arm, so as not to protrude radially outwardly from a perimeter defined by the arm; having a smoothed outer profile which potentially reduced damage to tissue the spacer comes in contact with.

FIGS. 14A-16 schematically illustrate structures of an imager (e.g. camera) insertable into the patient body and usable with systems and/or methods described herein, according to some embodiments.

In some embodiments, one or more imagers are inserted into the patient's body to provide vision during the surgical procedure.

In some embodiments, the imager is mounted on a distal end of a arm by which it is delivered, or is configured to extend from a distal end of a cannulated arm.

In some embodiments, in which surgeries performed via vaginal access, it may be desired to retroflect the arm of the imager, for example so as to obtain a field of view similar to the view obtained during abdominal procedures (such as procedures performed from a standard abdominal port). In some embodiments, the imager is directed and/or orientated inside the body in a manner which reduces or avoids blocking of the field of view, for example by surrounding tissue or organs. In an example, the colon may interfere and block the view, and it may be desired to push it away from the imager and/or to direct the imager such that it avoids the colon tissue.

In some embodiments, the camera is mounted on a flexible extension or arm. Optionally, the flexible extension or arm is constructed of a plurality of links or segments, which are bendable with respect to each other. A potential advantage of a flexible imager may include the ability to move the imager away from obstacles, and/or to provide multiple visual reference points to the user (e.g. surgeon). Obtaining multiple visual reference points may assist in situations in which the vision is unclear, for example due to large homogenous tissue (such as the inner wall of the abdomen) which may confuse the user with regards the current anatomical position.

FIGS. 14A, 14B and 14C illustrate an exemplary imager construction comprising a cannulated arm 102 formed with a side window 1104 from which a flexible (optionally telescopic) extension 1310 is configured to extend. In some embodiments, camera 1325 is mounted on a distal end of the extension 1310. In some embodiments, the extension 1325 can be advanced outwardly from the window 1104, and then set at a selectable angle β with respect to a centerline CL_{ARM} of the elongate arm 102. Angle β may range between, for example, 0-150°, such as 30°, 60°, 90 degrees or 120°. In some embodiments, as shown for example in FIG. 14A, extension 1310 is retroflected, positioning the camera 1325 such that it faces a proximal direction. In FIG. 14C, extension 1310 is fully received within and aligned with the arm 1102.

A potential advantage of a window 1104 formed along the wall of the arm 102, optionally in proximity to a distal end 1103 of the arm 102, may include that the camera 1325 can be advanced outwardly to obtain vision of a space located distally ahead of the arm 102, and/or obtain vision of a space located proximally to the arm 102. Positioning the camera 1325 radially outwardly with respect to the arm 102 may assist a user in directing (e.g., advancing and/or retracting) the arm 102.

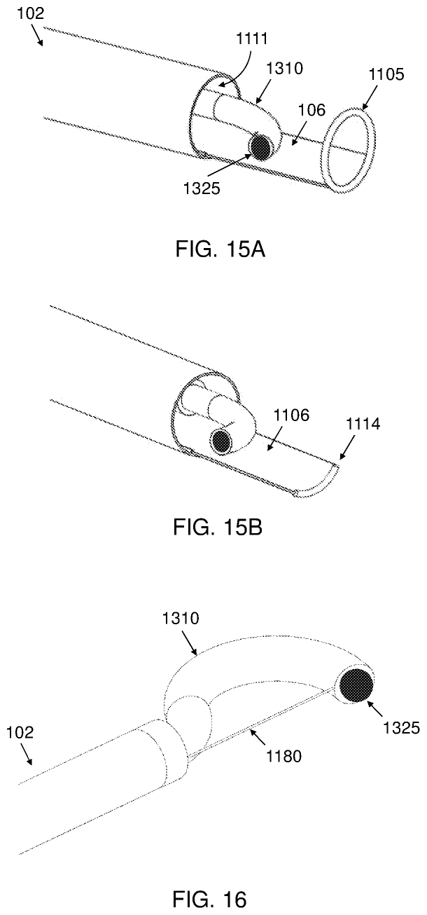

FIGS. 15A and 15B illustrate another exemplary imager construction, including an arm 102 comprising a distal spacer 1106, according to some embodiments. In some embodiments, spacer 1106 is aligned with respect to a wall of arm 102 and can be advanced or retracted from the arm opening 1111.

In some embodiments, as shown in FIG. 15A, spacer 1106 ends with a ring 1105; alternatively, as shown in FIG. 15B, spacer 1106 ends with an arc 1114. In some embodiments, ring 1105 is co-aligned with the arm 102. Optionally, the camera 1325 is advanceable through the ring 1105 or with respect to the arc 1114.

In some embodiments, camera 1325 is mounted on a distal end of a flexible extension 1310, which is configured to protrude outwardly from the arm 102, optionally to an extent defined by spacer 1106.

In some embodiments, during use, spacer 1106 is advanced distally of arm 102 to clear a working space for the camera 1325, such as by pushing away tissue.

FIG. 16 illustrates yet another exemplary imager construction, including an arm 102 from which a flexible extension 1310 comprising the camera 3125 is configured to extend, according to some embodiments. In some embodiments, a tensile element such as a cable 1180 (e.g., a wire cable) extends from a distal end of the arm 102 to a distal portion of flexible extension 1310. Optionally, cable 1180 is configured to be pulled on and/or or released from a proximal end of the arm (not shown herein), thereby approximating the distal end of the extension to the distal end of the arm, resulting in curvature of the extension 1310, for example to an S-shaped curve, a C-shaped curve, and the like. A potential advantage of curving the extension 1310 may include reducing a bending radius of the extension, may assist in maneuvering the imager inside the body, for example when rotating the camera 1325 with respect to a long axis defined by the arm 102.

### Additional Discussion of Embodiments

According to an aspect of some embodiments there is provided a method for accessing a treatment zone through the vagina, comprising: selecting an entry angle; positioning a surgical port adjacent the vaginal opening; introducing a trocar through the surgical port; introducing a cannula through the trocar and advancing the cannula such that the cannula enters through a posterior wall of the vagina; externally supporting the port, trocar and cannula assembly using an extension, the extension attached on one end to at least one component of the assembly and on the other end to a fixed, stable reference; the extension holding the assembly at a fixed position in which the trocar and cannula are slanted at the selected entry angle.

In some embodiments, the fixed reference comprises a surgical bed on which the patient lays.

In some embodiments, the extension comprises an adjustable fixation arm.

In some embodiments, the method comprises, prior to selecting, identifying a location of the Pouch of Douglas, and selecting the entry angle according to the location.

In some embodiments, holding comprises locking the assembly in position with respect to at least one of: the patient body and the surgical bed.

In some embodiments, supporting comprises reducing a weight load on the posterior wall of the vagina by shifting the weight of the assembly onto the extension.

In some embodiments, the method further comprises introducing one or more surgical arms through the cannula.

According to an aspect of some embodiments there is provided a method of controlling via one or more input device, one or more surgical mechanical arms insertable into a body of a patient, each surgical mechanical arm comprising a plurality of movable joints; the method comprising: in a first mode of operation, navigating the surgical mechanical arms into the patient's body; in a second mode of operation, carrying out surgical acts using the surgical mechanical arms; wherein in the first mode of operation movement of each of the surgical mechanical arms is restricted to movement of a single joint out of the plurality of joints, and to linear movement of the surgical mechanical arm as a single unit.

In some embodiments, navigating comprises retroflecting the surgical mechanical arms within the patient's body.

In some embodiments, in the first mode of operation the surgical mechanical arms are controlled by thumb operated input.

In some embodiments, in the second mode of operation the surgical arms are controlled by avatar input arms.

In some embodiments, the surgical mechanical arms are controlled by haptic handles during both the first and the second modes of operation.

In some embodiments, in the first mode of operation manipulation of the input device by a user is translated to a speed of movement of the surgical mechanical arm, and in the second mode of operation displacement of the input device by the user is translated to a relative displacement of the surgical mechanical arm.

In some embodiments, in the second mode of operation a clutch-like mode is enabled, disconnecting control of the surgical arm by the one or more input device.

In some embodiments, there is provided a control console for control of one or more surgical mechanical arms, comprising: thumb operated input for controlling the first mode of operation; hand operated input for controlling the second mode of operation; and a screen interface.

In some embodiments, the thumb operated input comprises a nipple engagable by the user's thumb; whereby pushing of the nipple from a central rest position actuates respective movement of the surgical mechanical arm; and wherein a speed of the movement is affected by an extent in which the nipple was pushed relative to its rest position.

In some embodiments, in the second mode of operation manipulation of the hand operated input by a user is translated to a similar articulation of the surgical arm.

According to an aspect of some embodiments there is provided a method for operating a patient, comprising: introducing one or more surgical arms through the vagina into the abdominal cavity; bending the one or more surgical arms to a retroflected position, wherein during introducing and bending, articulation of the one or more surgical arms is limited to linear movement and to movement of a single arm joint only; and operating within the abdominal cavity using the one or more surgical arms in the retroflected position.

In some embodiments, wherein during linear movement the surgical arm moves as a single unit, and wherein movement of a single arm joint comprises flexion and extension of an elbow joint.

According to an aspect of some embodiments there is provided a method of controlling via one or more input device, one or more surgical mechanical arms insertable into a body of a patient, each surgical mechanical arm comprising a plurality of movable joints; the method comprising: setting a level of force resistance to be sensed by a user manipulating the one or more input device, wherein the level is selected according to at least one of: a current anatomical location of the one or more surgical arms, a current articulated position of the one or more surgical arms; and providing the set level of force resistance in response to manipulation of the one or more input device by a user.

In some embodiments, the method comprises increasing the level of force resistance when manipulation of the one or more input device generates a surgical arm movement which is not mechanically supported by the one or more surgical arms.

In some embodiments, the method comprises increasing the level of force resistance when manipulation of the one or more input device generates a movement which is adjacent or at disallowed anatomical areas.

According to an aspect of some embodiments there is provided a method of controlling via one or more input device, one or more surgical mechanical arms insertable into a body of a patient, each surgical mechanical arm comprising a plurality of movable joints; the method comprising: generating movement of the one or more surgical arms only if a respective control manipulation of the one or more input device comprises of a similar movement performed by the user, the user movement required to be scaled up by a preselected coefficient which is larger than 1.

According to an aspect of some embodiments there is provided an imager for insertion into the body of a patient, comprising: an elongate shaft; a flexible extension positioned within the shaft and extendible outwardly from the shaft; and a camera mounted onto a distal end of the flexible extension, wherein at least one of the camera and the flexible extension are positionable at a retroflected position which directs the camera backwards, opposite the direction of advancement of the elongate shaft.

In some embodiments, the elongate shaft comprises a window at a side wall of the shaft, the flexible extension configured to protrude outwardly from the window and to position the camera at an angle with respect to a longitudinal axis of the shaft.

In some embodiments, the imager is insertable into the body along with one or more surgical arms, and at least the flexible extension is configured to be retroflected along with the surgical arms.

In some embodiments, the elongate shaft comprises a tissue spacer extending from a distal end of the shaft, the tissue spacer ending with a ring through which the flexible extension can pass.

In some embodiments, the imager comprises a tensile element extending between a distal end of the flexible extension and the shaft, the tensile element configured to be pulled on to change a curvature of the flexible extension.

According to an aspect of some embodiments there is provided an imager for insertion into the body of a patient, comprising: at least two cameras mounted on a 3-axis gimbal support head, the gimbal support head extending from a flexible shaft; wherein the gimbal support head is configured for pivoting with respect to the flexible shaft to position the at least camera such that the camera faces backwards, in a direction of the flexible shaft; and wherein the at least two cameras provide 3 dimensional vision.

According to an aspect of some embodiments there is provided an imager for insertion into the body of a patient, comprising: a flexible shaft comprising a rigid extension at a distal end of the shaft; the rigid extension defining a recess in which a pivotable head is received, the pivotable head including a camera; wherein the pivotable head is configured for pivoting within the recess from a position in which the head is flush with the rigid extension to a position in which the head is at angle of between 1-135 degrees with respect to the rigid extension.

According to an aspect of some embodiments there is provided a method for operating a patient, comprising: introducing one or more surgical arms through the vagina into the abdominal cavity; bending the one or more surgical arms to a retroflected position; wherein during the introducing and the bending, articulation of the one or more surgical arms is limited to linear movement and to movement of a single arm joint only; and operating within the abdominal cavity using the one or more surgical arms in the retroflected position.

In some embodiments, in the linear movement the surgical arm moves as a single unit, and wherein movement of a single arm joint comprises flexion and extension of an elbow joint.

According to an aspect of some embodiments there is provided a method of controlling via one or more input device, one or more surgical mechanical arms insertable into a body of a patient, each surgical mechanical arm comprising a plurality of movable joints; the method comprising: setting a level of force resistance to be sensed by a user manipulating the one or more input device, wherein the level is selected according to at least one of: a current anatomical location of the one or more surgical arms, a current articulated position of the one or more surgical arms; and providing the set level of force resistance in response to manipulation of the one or more input device by a user.

In some embodiments, the method comprises increasing the level of force resistance when manipulation of the one or more input device generates a surgical arm movement which is not mechanically supported by the one or more surgical arms.

In some embodiments, the method comprises increasing the level of force resistance when manipulation of the one or more input device generates a movement which is adjacent or at disallowed anatomical areas.

According to an aspect of some embodiments there is provided a method of controlling via one or more input device, one or more surgical mechanical arms insertable into a body of a patient, each surgical mechanical arm comprising a plurality of movable joints; the method comprising: generating movement of the one or more surgical arms if a respective control manipulation of the one or more input device comprises of a similar movement performed by the user, the user movement required to be scaled up by a preselected coefficient which is larger than 1.

According to an aspect of some embodiments there is provided an imager for insertion into the body of a patient, comprising: at least two cameras mounted on a 3-axis gimbal support head, the gimbal support head extending from a flexible shaft; wherein the gimbal support head is configured for pivoting with respect to the flexible shaft to position the at least camera such that the camera faces backwards, in a direction of the flexible shaft; and wherein the at least two cameras provide 3 dimensional vision.

According to an aspect of some embodiments there is provided an imager for insertion into the body of a patient, comprising: a flexible shaft comprising a rigid extension at a distal end of the shaft; the rigid extension defining a recess in which a pivotable head is received, the pivotable head including a camera; wherein the pivotable head is configured for pivoting within the recess from a position in which the head is flush with the rigid extension to a position in which the head is at angle of between 1-135 degrees with respect to the rigid extension.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as collecting dental measurements, might be expected to use completely different methods, e.g., making use of expert knowledge and/or the pattern recognition capabilities of the human brain, which would be vastly more efficient than manually going through the steps of the methods described herein. An aspect of some embodiments relates to a surgical procedure including a setup for insertion of one or more surgical arms through the vagina. In some embodiments, preparation for vaginal access comprises coupling a port element between the patient's legs, adjacent an entry to the vagina, positioning a trocar through the port, and introducing a cannula through the trocar, positioning the cannula through an access cavity produced at the vaginal wall, for example, at a wall of the posterior vaginal fornix.

In some embodiments the port, trocar and cannula assembly is held and supported in position by one or more external mechanical means, for example, a fixation arm which maintains the assembly in a selected position and orientation (e.g. a selected entry angle, a selected depth). In some embodiments, the fixation arm extends from the assembly to a fixed, stable reference, for example the surgical bed on which the patient lays. In some embodiments, a positioning angle of the assembly is selected so that the surgical arms, when introduced through the cannula, are directed towards the Pouch of Douglas. In some embodiments the angle is selected to that anatomical areas which are not targeted by the operation, for example, the rectum and/or uterus, are avoided.

In some embodiments, the fixation arm is not attached directly to the assembly, and instead coupled to an instrument holder which extends from the assembly. In an example, the fixation arm extends from a mounting block of the instrument holder.

In some embodiments, the fixation arm is adjustable, for example providing for setting the assembly at a selected height, orientation and/or slanting angle with respect to the vaginal opening and/or with respect to the bed and/or with respect to a motor unit which actuates movement of one or more surgical arms introduced through the cannula. In some embodiments, adjustment is via one or more: flexible joints of the fixation arm, one or more knobs of the fixation arm, for locking segments in position, adjustable segments of the fixation arm, for example, telescopic shafts, and/or other adjustable means.

A potential advantage of fixating the port, trocar and cannula assembly in position and mechanically supporting the assembly may include one or more of: reducing or avoiding mechanical stress on the thin vaginal wall (which is only a few mm thick); reducing or avoiding slipping of the assembly or components thereof into the vagina; reducing or avoiding slipping of the assembly or components thereof out from the vagina, shifting the weight away from vaginal wall and the like. Another potential advantage of fixating the assembly may include reducing or preventing movement of the assembly, for example when insufflation of the patient's abdomen is performed.

In some embodiments, preparation for vaginal access comprises creating access to the abdomen via the Pouch of Douglas. In some embodiments, access is created by penetrating the posterior vaginal wall with a needle, and dilating the punctured hole, optionally with a dedicated dilator, at least to an extent sufficient for introducing of the cannula through which the surgical arms are inserted.

In some embodiments, prior to introducing the surgical arms, the surgical arms are aligned and positioned with respect to the vaginal access port, for example, by setting a position of a motor unit of the surgical arms (e.g. with the aid of a motor unit fixation arm).

In some embodiments a camera is introduced through the vaginal access path, to provide vision during the operation. Additionally or alternatively, a camera is introduced through the umbilicus, and directed to provide vision of the vaginal access path.

Abdominal vision provided by a camera inserted through the umbilicus may be advantageous for surgeons who are more familiar with an abdominal view (for example as compared to vaginal access view), such as laparoscopic surgeons.

In some embodiments, surgical arm insertion is performed gradually, for example so that one or more selected segments of the surgical arm protrude from cannula (e.g. the surgical arm is advanced inwardly up to the wrist joint, then up to the elbow joint, and so on). In some embodiments, advancement of the arms is limited by a stopper.

A general aspect of some embodiments of the invention relates to control of one or more surgical mechanical arms using a first mode of operation for navigation of the arms to a selected location and position within the patient's body, and a second mode of operation for performing surgical acts within the selected location. In some embodiments, control at the first mode involves translating a user (e.g. surgeon) manipulation of an input arm (e.g. an avatar arm, a joystick) into a respective articulation of the surgical arm, whereby the speed of movement of the surgical arm changes as the extent of manipulation of the input arm relative to a rest position of the input arm changes.

In some embodiments, control at the second mode involves translating a user manipulation of an input arm into a respective position of the surgical arm, for example so that the position is set directly according to the input arm position (e.g. identical articulation). In some embodiments, a user's displacement of the input arm is translated to a relative displacement command to the surgical arm. In some embodiments, the second mode includes a different control, for example one in which a user's extent of manipulation of the input arm is in a different proportion to similar movement carried out by the surgical arm.

In some embodiments, movement of the surgical arm at the first mode is restricted, for example providing only for flexion and/or extension of an elbow joint of the surgical arm, and for linear movement of the surgical arm as a single unit. Optionally, other joints of the surgical arm (e.g. shoulder joint, wrist joint) are held stationary, optionally locked in position. Alternatively, at least a partial, limited movement of one or more other joints is allowed.

In some embodiments, introducing of the surgical arms into the body (e.g. through the vagina), navigation of the arms (e.g. into the abdomen) and optionally retroflection of the surgical arms is carried out when the surgical arms are controlled using the first operation mode. A potential advantage of restricting articulation of the surgical arm during introducing of the arm and/or during retroflection of the arm may include reducing a bending radius of the surgical arm, thereby reducing a likelihood of encountering surrounding obstacles such as the inner abdominal wall. Another potential advantage of restricting articulation of the surgical arm during navigation and/or retroflection processes may include improved control over the surgical arms. In some embodiments, the first operation mode (speed control mode) allows a user to continuously control movement of the surgical arms, even during retroflection of the arms, when relative directions (e.g. upwards and downwards) are reversed.

In some embodiments, a system configured for controlling the surgical arms at both modes includes dual control means, including two different sets of controls. In some embodiments, a first control set is used for insertion of the surgical arms into the body, and a second control set is used for performing surgical acts inside the body. Alternatively, both sets are used for at least one of the stages (insertion and surgery); alternatively, only one set is used for both stages.

In some embodiments, the first mode is controlled by a set of thumbsticks. Optionally, an extent of pushing of a nipple of each thumbstick relative to a central rest position of the nipple affects a speed in which an arm articulation takes place. Optionally, the nipple springs back to its rest position when the user lifts their thumb. In some embodiments, the second operation mode is controlled using a set of avatar input arms, manipulated by the user's hands.

Additionally or alternatively, a set of haptic handles (including for example two handles, one for control of each surgical arm) are used for carrying out both modes of operation. Optionally, at the first mode of operation, the handles are set to provide counter resistance to user movement, optionally spring-like resistance, in response to moving the arm away from its rest position. Optionally, at the second mode of operation, the haptic handles are set (for example pre-programmed) to provide selected or changing resistance in response to manipulation by the user.

In some embodiments, control of surgical arm movement is according to predefined algorithms. In some embodiments, control algorithms are stored in a system memory, and/or on a remote server in communication with the surgical system. In some embodiments, control algorithms are implemented in system circuitry, for example, the system controller is pre-programmed with one or more control algorithms. Optionally, selection of one or more control algorithms to be implemented is by a user, for example via a system user interface. Additionally or alternatively, automatic selection is performed by the system, for example by sensing a current position of the surgical arms, by vision of a current anatomical area in which the surgical arms operate, and/or other.

In some embodiments, an algorithm is defined to provide various (and/or varying) resistance levels to the user, for example to prevent movement which is not supported by the surgical arm and/or to prevent or restrict movement into or towards disallowed anatomical areas and/or to indicate to a user a type or tissue or organ being contacted. In some embodiments, an algorithm is defined to provide the user with sensible feedback upon manipulation of the input arm, for example, to provide the user with wall-like resistance in response to movement that should be avoided, and no or float-like resistance in response to desired or allowed movement. The resistance may be selected according to one or more of: the type of movement, the current articulation of the surgical arm; the current anatomical position of the surgical arm; the extent of manipulation.

In some embodiments, a custom algorithm is defined to scale the user's motion, such as to increase accuracy of movement. Such scaling may include amplifying the movement required on the user end by a selected coefficient (optionally larger than 1, larger than 2, larger than 5) to produce a similar non-amplified movement of the surgical arm. Optionally, surgical arm movement is performed at a selected proportion to user movement.

In some embodiments, a custom algorithm is defined to filter certain signals, for example filter out user hand tremors.

An aspect of some embodiments relates to an imager (e.g. a camera) for use during surgery, the imager configured for retroflection inside the body while potentially avoiding blockage of the field of view by adjacent organs. In some embodiments, the camera itself is configured to be retroflected, for example positioned at an angle with respect to shaft from which it extends; additionally or alternatively, the shaft from which the camera extends is flexible and can be bent to position the camera to face backwards (e.g. opposite the direction of advancement of the shaft into the body).

In some embodiments, the imager is configured for obtaining multiple visual reference points, for example by the user directing the camera at a plurality of directions, which may assist the user in determining a current anatomical position. In some embodiments, a camera is mounted onto a flexible extension configured to be advanced (and/or retracted) through a cannulated shaft. In some embodiments, the shaft is formed with a window, for example at a side wall of the shaft, and the flexible extension can be pushed at least partially out the window to position the camera at a selected location and/or angle. Optionally, by manipulation of the flexible extension on which the camera is mounted, the camera can be turned (e.g. retroflected), for facing a direction opposite the direction of advancement of the shaft. Optionally, a field of view obtained by the retroflected camera includes a working space in which the retroflected surgical arms operate.

In some embodiments, the flexible extension is positionable at an angle with respect to longitudinal axis defined by the shaft. In some embodiments, a far end of the flexible extension, onto which the camera is mounted, is coupled to a tensioning element, for example a wire cable, which extends to a distal opening of the shaft. Optionally, upon pulling or releasing of the cable, a curvature of the flexible extension is changed. In an example, the extension is curved into an S-shaped curve, a C-shaped curve. A potential advantage of approximating the camera by manipulating a curvature of the flexible extension may include reducing a bending radius of the extension, thereby potentially facilitating turning or generally maneuvering the camera inside the patient body. In some embodiments, the shaft through which the camera is advanced comprises a distal spacer for clearing a working space distally of the shaft, for example by pushing away tissue. Optionally, the spacer comprises a ring or arc at its distal end. Optionally, the camera is advanceable through the ring or with respect to the arc. Potential advantages of a ring or arc configured at a distal end of the spacer may include: being aligned with a circumference of the shaft, so as not to protrude radially outwardly from a perimeter defined by the shaft; having a smoothed outer profile which potentially reduced damage to tissue the spacer comes in contact with.

Some embodiments comprise a camera mounted on a head configured for pivoting with respect to a shaft from which it extends. In some embodiments, the shaft comprises a distal rigid extension which defines a recess in which the head is received, and from which it can be pivoted outwardly, for example to position the camera at an angle relative to the rigid extension (e.g. an angle of between 5-150 degrees—for example, between 5-135 degrees). Optionally, when the head is received within the recess, it is flush with the rigid extension.

In some embodiments, a gimbal support is provided, and one or more cameras are mounted onto the gimbal support. Optionally, the gimbal support is configured for rotating with respect to a shaft from which it extends, to position the one or more cameras at a selected angle and/or orientation.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is noted that the order and/or structure of steps described hereinbelow, especially in the description of the surgical use flow, is optional. Some embodiments may be carried out by performing only a part of the steps and/or performing a step only in part.

### General Use Flow

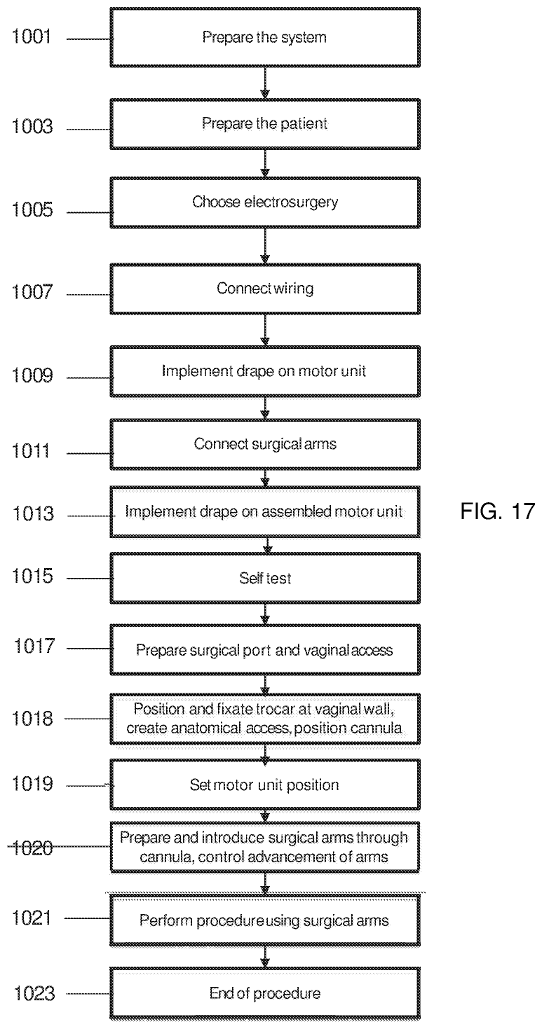

FIG. 17 is a flowchart of general use of a medical robotic surgical system, according to some embodiments.

Use flow as described herein may be implemented, in some embodiments, during surgical operations performed by accessing the body via one or more natural orifices such as the vagina, anus, mouth, esophagus, windpipe, nostril, ear canal. In the exemplary flowchart, the process is described with respect to procedures performed by accessing through the vagina.

At 1001, the surgical robotic system is prepared. In some embodiments, system preparation involves setting electrical connections, such as: connecting the control console to powering means (mains power, a battery, and/or other powering means); connecting the motor unit to the control console; connecting the motor unit to an electrosurgery generator.

In some embodiments, system preparation involves a mechanical setup, for example: attaching a fixation arm to the surgical bed; setting a position of the fixation arm (e.g. by adjusting fixation arm joints, for example via knobs); locating a motor unit on the fixation arm. Additionally or alternatively, if a motor unit cart is used, the motor unit position is adjusted (e.g. in height and/or angular position); the cart is wheeled to a selected location relative to the surgical bed.

At the end of system preparation, the motor unit, control console and/or electrosurgery generator are turned off, before being turned on again at the initiation of the procedure.

At 1003, the patient is prepared. In some embodiments, the patient is at least partially covered with a surgical sterile drape. In some embodiments, the patient is anesthetized before the operation. In some embodiments, the patient's legs are placed on leg stirrups. In some embodiments, the surgical bed is tilted, for example lowering the patient head side, so as to facilitate access through the vagina. In some embodiments, the patient is positioned such that the buttocks extend slightly from the surgical bed, for example about 2 cm or about 2.5 cm away from the table.

At 1005, an energy type is selected for each of the surgical arms (bipolar or monopolar energy) and at 1007 the selected wiring is connected. Optionally, energy type selection is performed via a screen interface of the control console. In some embodiments, wiring includes connecting cables between the electrosurgery generator and the motor unit.

At 1009, in some embodiments, a surgical sterile drape is implemented on the motor unit. In some embodiments, in which a two-portion drape comprising a bottom drape sheet and a top drape sheet is used, the bottom drape sheet is deployed on the motor unit, leaving exposed access to attachment of the surgical arms to the motor unit.

At 1011, in some embodiments, one or more surgical arms (e.g. 2, 3, 4, 6, 8 or intermediate, larger or smaller number) are taken out from their sterile cover (e.g. from a sterile pouch) and connected to the motor unit. Optionally, the surgical arms are assembled on the motor unit via one or more access doors configured on the motor unit. Optionally, once a surgical arm is assembled, closure of the access door locks the surgical arm in place. In some embodiments, arm assembly is carried out by holding on to one or more handles configured on the arm, and inserting the arm through the respective access door on the motor unit into a designated slot.

At 1013, in some embodiments, a drape is implemented on the assembled motor unit. In an example in which a two-portion drape is used, a top drape is deployed on the motor unit, covering remaining exposed portions of the motor unit.

At 1015, in some embodiments, a self-test is performed. In some embodiments, the self test is performed automatically by the system (for example controlled by the system controller). Optionally, during the self-test, the one or more surgical arms are moved. In some embodiments, input arms are tested (e.g. moved) by the user, optionally according to instructions. In some embodiments, articulation of the surgical arms during the test is restricted, for example, to less than a maximal movement range. In some embodiments, during the self test, calibration (e.g. of input arm position and/or orientation with respect to surgical arm position and/or orientation) is tested. In some embodiments, during the self test, communication between system components (e.g. input arms and surgical arms, control console and surgical arms, electrosurgical generator and surgical arms and/or others) is tested. In some embodiments, during the self test, voltage levels are tested. In some embodiments, during the self test, operation of one or more system sensors is tested. In some embodiments, during the self test, operation of one or more motors, drivers and/or other actuators of the system are tested.

The progress and/or results of the self test and/or the instructions for operating the input arms may be presented to the user on the screen interface of the control console.

At 1017, in some embodiments, a surgical port and/or vaginal access are prepared. In some embodiments, preparation involves coupling a port element (e.g. a laparoscopic port) to the patient's body at a selected insertion point, for example adjacent an entry to the vagina, externally to the body.

In some embodiments, an incision is made. In some embodiments, the position of the port is selected according to the type of procedure being performed, a desired anatomic analysis and/or according to a desired view for inspecting the operated area using a camera insertable through the port.

At 1018, in some embodiments, a trocar is positioned at the port, for example to allow for insertion of a cannula, one or more surgical arms, a camera, one or more tools (such as suction, irrigation tools) into the patient body.

Optionally, in some embodiments, a camera is introduced through the umbilicus, and set to allow vision of the vaginal access point from the direction of the umbilicus. Insertion of a camera through the umbilicus may be especially advantageous in specific procedures and/or for surgeons who are less familiar with vaginal access and are used to operating according to an abdominal view, such as laparoscopic surgeons.

In some embodiments, locating a trocar at a wall of the vagina (in some embodiments, at a wall of the posterior vaginal fornix), which has a thickness of a few millimeters only, for example, between 1-2 mm requires external means for holding the trocar, for example so as avoid one or more of: mechanical stress on the vaginal wall; slipping of the trocar deeper into the vagina; slipping of the trocar out of the vagina. Therefore, in some embodiments, a fixation arm (e.g. GYN fixation arm, as referred to below) is positioned to hold and fixate the trocar, optionally with respect to a stable reference point, such as the surgical bed. In some embodiments, the trocar is fixated at a selected orientation. Optionally, a positioning angle of the trocar is selected as one which avoids, in one direction, encountering the rectum, and in the opposite direction, encountering the uterus.

In some embodiments, the trocar is positioned at an angle directed towards the pouch of Douglas. Optionally, the trocar is fixated in a position in which a distal end of the trocar extends about 1 cm, 1.5 cm, 2 cm, 2.5 cm, 3 cm, 3.5 cm, 4 cm from the vagina into the abdomen.

In some embodiments, the patient's abdomen is insufflated. Optionally, insufflation does not affect the trocar position at the vagina.

In some embodiments, a dilator is inserted through the trocar. Optionally, a needle is then inserted through the dilator to penetrate the vaginal wall for producing access, for example, penetrating the wall to enter the pouch of Douglas. At this stage, the dilator, guided by the needle, is advanced inwards for widening the punctured hole. In some embodiments, a cannula is then placed through the trocar. Optionally, dilation of the punctured hole by the dilator is to an extent which matches a size (e.g. diameter) of the cannula.

At 1019, in some embodiments, the motor unit position is set so as to introduce the surgical arms through the vagina. In some embodiments, a position of the motor unit with respect to the vaginal cavity is adjusted, for example by modifying a fixation arm, cart and/or other support on which the motor unit is positioned. Optionally, a height, angular position and/or distance of the motor unit from the vaginal cavity are adjusted. In an example, one or more of a height, distance, relative location between the patient's legs, pitch, yaw, and/or roll of the motor unit with respect to the vagina, for example with respect to the vaginal opening at which the port is positioned, are adjusted. In some embodiments, the motor unit is coupled to its support via a slidable coupling, for example by a dovetail coupling configured to slide in a respective recess on the support. In some embodiments, the motor unit is locked in a selected position, for example via one or more latches.

At 1020, in some embodiments, the surgical arms are prepared for insertion by covering at least a segment of each arm, for example a flexible segment of the arm, by a sheath. Optionally, the sheath is formed with a stopper, for example in the form of an external protrusion, which limits advancement of the sheath within the cannula, so that the surgical arms may continue to extend externally from the cannula without being covered by the sheath.

In some embodiments, the surgical arms are introduced into the cannula with the aid of an instrument handle, which includes, for example, an insertion and/or alignment and/or spacing handle, and a mounting block. In some embodiments, a distal end of the handle is telescopic, so that sliding proximally of a handle portion gradually exposes one more selected segments of the surgical arms, for example, up to the wrist joint, up to the elbow joint. In some embodiments, advancement of the arm is limited by a stopper, for example a stopper of the handle. Additionally or alternatively, in some embodiments, the surgical arm is advanced until advancement of its respective motor unit is stopped, for example by a stopper on the motor unit fixation arm.

In some cases, a distance of advancement of the surgical arm into the body is selected so as to avoid contact of the distal end of the surgical arm (e.g. the arm end-effecter) with tissue of surrounding organs. In some embodiments, the handle aligns between the surgical arms and the cannula during introducing of the arms into the body. In some embodiments, the arms are advanced into the body until an end-effecter (e.g. tissue grasper) is visible on the user interface screen.

At 1021, the procedure is carried out by a surgeon controlling the surgical arms via the input arms at the control console. In some embodiments, the procedure involves one or more navigating the surgical arms to a selected position, grasping tissue, dissecting tissue, moving tissue, suturing, and/or other surgical acts. In some embodiments, the arms are navigated to a selected position and then retroflected. A potential advantage of retroflection may include positioning the surgical arms at an orientation in which the surgeon is more comfortable and/or familiar with for performing the procedure. Another potential advantage may include carrying out laparoscopic surgical tasks from a laparoscopic point of view, yet without incising the abdominal wall (potentially avoiding scarring and/or other undesired effects of an abdominal incision.) Another potential advantage of modifying the relative position by retroflecting the arms may include shortening a learning curve of surgeons when carrying out surgical operations such as laparoscopic operations using the system.

In some embodiments, electrosurgery is applied. Optionally, when applying energy to the surgical arms, the energy type is verified by the surgeon, for example by interacting with the screen interface. In some embodiments, applying of energy is actuated by pressing a footpedal on the control console.

In some embodiments, the system may be paused and/or resumed during operation, for example by pressing a button on the on the input arm and/or by touching the screen interface.

In some embodiments, in case of an emergency (e.g. mechanical failure, power loss), an emergency button may be pushed, stopping movement of the surgical arms, for example by stopping the motorized actuation of the arms. Optionally, manual manipulation (e.g. release of graspers to release tissue, articulation of an arm joint) and/or manual removal of the surgical arms from the body is enabled. In some embodiments, manual release straightens the surgical arms to facilitate pulling the arms out of the body. In some embodiments, by ensuring that the surgical arms operate within an anatomical space that provides leeway, damage to tissue during emergency release is reduced or prevented. In some embodiments, in the case of power loss, one or more breaks lock the system to prevent undesired movement.

At 1023, the procedure ends. In some embodiments, the surgical arms are brought to a null (rest) position and are simultaneously extracted from the body. In some embodiments, the “pause-resume” button is pushed before complete withdrawal of the surgical arms. In some embodiments, following withdrawal of the surgical arms, the camera remains inside the patient's body, for example to inspect for bleeding. The camera, trocar, port can then be removed from the body, and the incision is closed. In some embodiments, an “end of procedure” button is pushed, for example on the screen interface, to allow releasing the surgical arms from the motor unit. Optionally, the locking doors are opened.

At this point, surgical drapes are removed from the motor unit, the motor unit and surgical arms are cleaned, cables are disconnected, and single use tools are disposed of.

It is noted that the above method is only an example of a surgical procedure carried out using the one or more surgical arms, and that the order of actions may change. The described actions may be carried out by one or more of: a sterile surgeon, a non-sterile surgeon, a sterile nurse, a non-sterile nurse, depending on the type of action performed.

It is noted that the above method or portions thereof can be implemented for various types of operations such as gynecologic, laparoscopic, otorhinolaryngology (ear, nose and throat) surgeries.

### Exemplary Surgical System

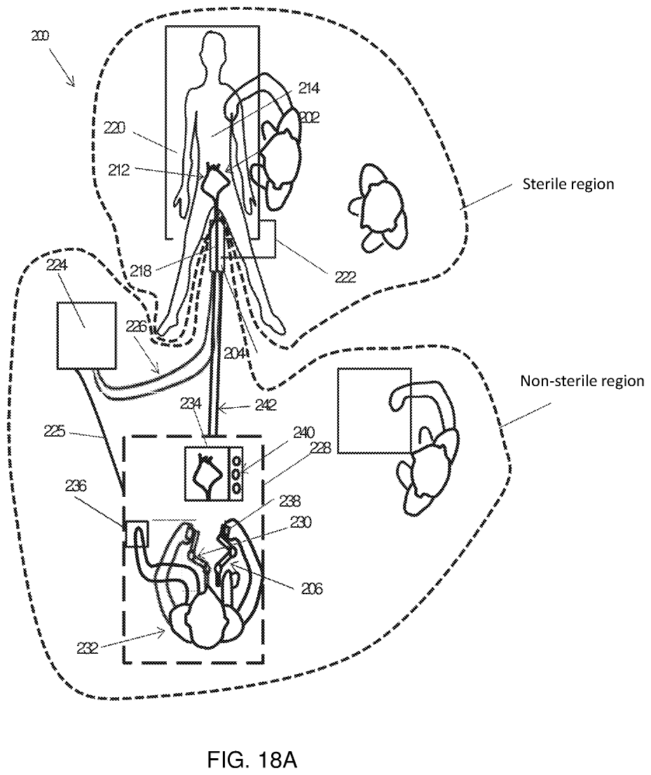

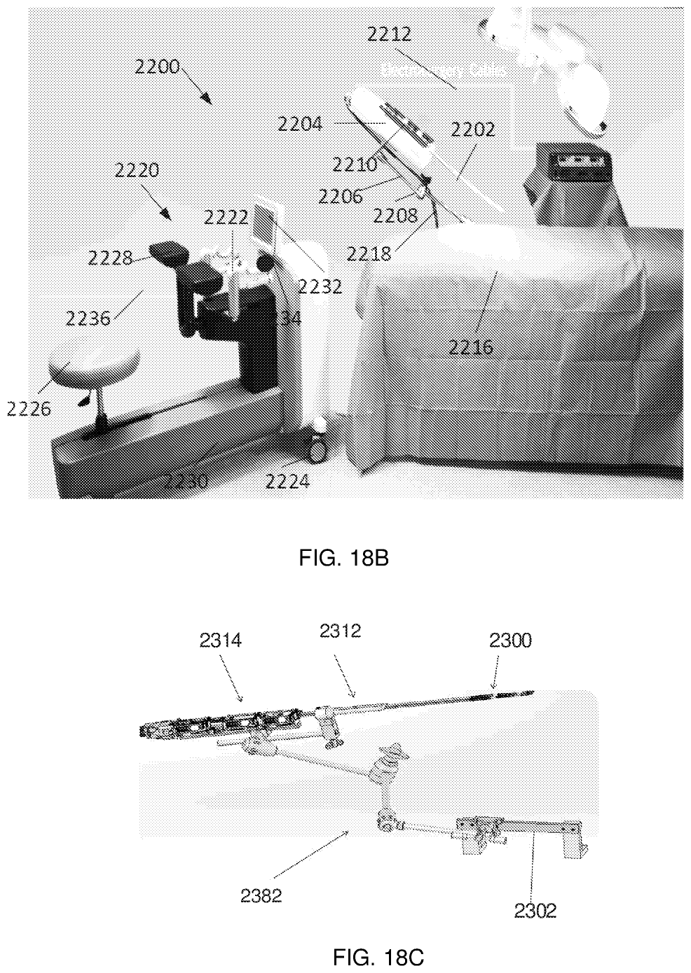

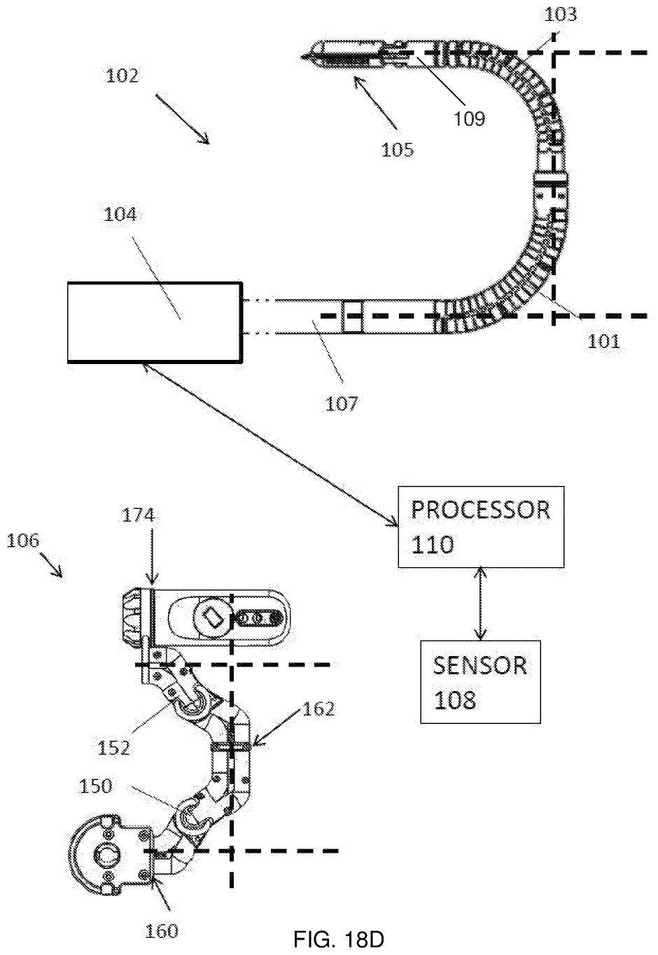

FIG. 18A is a simplified schematic drawing of a surgical system 200, according to some embodiments.

In some embodiments, surgical system 200 includes at least one surgical mechanical arm, for example, a plurality of surgical mechanical arms 202, 212 e.g. two surgical mechanical arms. In some embodiments, surgical mechanical arms are sized and/or shaped for insertion into a human patient's body 214.

In some embodiments, the system includes at least one motor unit, for example, a plurality of motor units 204, 216, where, in some embodiments, each of surgical mechanical arms 202, 204 is actuated by a motor unit. For example, where a first surgical arm 202 is actuated by a first motor unit 204 and/or a second surgical arm 212 is actuated by a second motor unit 218.

In some embodiments, one or more motor unit and/or one or more surgical arm is attached to a patient support surface 220 (e.g. a bed), for example by a support 222. In an exemplary embodiment, one or more motor unit is attached to patient support surface 220. A potential benefit of the device being coupled to a bed is the ability to move and/or change an angle of the bed, for example, during surgery, while the device remains in the same position relative to the bed and/or patient. Alternatively, or additionally, in some embodiments, a device position with respect to the patient and/or the bed is adjustable, for example, before treatment with the device and/or during surgery.

In the example shown in FIG. 18A, patient 214 is illustrated in a suitable position for insertion of the device into and/or through the vagina (and/or anus and/or undercarriage), where, for example, the patient's legs are apart (e.g. elevated and/or held apart e.g. held by stirrups which are not illustrated).

In some embodiments, surgical arms 202, 212 are controlled (e.g. by a user 232) at a control console 228. In some embodiments, movement of surgical arms 202, 212 is controlled. In some embodiments, electrosurgical charging of arms 202, 212 is controlled. In some embodiments, one or more motor unit (e.g. each motor unit 204, 218) is connected to control console via data and/or electrical supply connections 242.

In some embodiments, control console 228 includes a plurality of user interfaces: In some embodiments, control console 228 includes one or more input arm 206, 230, where the control console is configured to generate control signals upon movement of the arm/s. The input arms may take the form of one or more of: joysticks, thumbsticks (thumb operated button), haptic handles. In some embodiments, a processor (not illustrated) generates control signals when input arm/s are moved (e.g. as described regarding sensor 108 and processor 110, FIG. 17). In some embodiments, one or more input arm includes an additional user interface (not illustrated), for example, one or more button and/or switch.

In some embodiments, control console includes a display 234 (e.g. a graphic user interface (GUI)). In some embodiments, display 234 is configured to display imaging of a surgical zone, for example, to display images collected by a camera inserted into patient 214 e.g. with surgical arms 202, 212. In some embodiments, display 234 is a touch screen configured to receive user input, potentially providing a user input.

In some embodiments, control console 228 includes one or more additional user interface 240 (e.g. button, switch) e.g. located on and/or near display 234.

In some embodiments, system 200 includes connectivity to and/or includes an electrosurgical generator 224. In some embodiments, for example, as known in the art of electrosurgery, electrosurgical generator 224 supplies high-frequency (e.g. radio frequency) alternating polarity electrical current. In some embodiments, electrosurgical generator 224 is configured to supply different frequencies and/or powers, for example, suitable for cutting and/or coagulating and/or desiccating and/or fulgurating tissue.

In some embodiments, electrosurgical generator 224 is a part of control console 228. Alternatively, in some embodiments, electrosurgical generator 224 is a separate device the control console and/or motor units, for example including connectivity to the electrosurgical generator. For example, in an exemplary embodiment, electrosurgical generator 224 is a Covidien Force FX ESU Electrosurgical Generator. In some embodiments, supply to the motor units is via cable/s 226 which are, for example, configured to transfer radio frequency electrosurgical power.

In some embodiments, one or more surgical mechanical arm 202, 212 is supplied with electrical power by a motor unit to which the arm is attached. In some embodiments, surgical arm/s are supplied (e.g. indirectly through motor unit/s) with power by electrosurgical generator 224.

In some embodiments, electrosurgical generator 224 includes one or more user interface e.g. for control of supply of electrosurgical power supply to arms 202, 212. In some embodiments, the electrosurgical generator is controlled by a control console user interface e.g. 234 and/or 240.

In some embodiments, control console includes a foot pedal 236. Alternatively or additionally, foot pedal 236 is provided as part of and/or attached to electrosurgical generator 224. In some embodiments, foot pedal 236 is connected via a data and/or power connection 225 to electrosurgical power generator 224. In some embodiments, foot pedal 236 controls supply of electrosurgical power to the surgical mechanical arm/s 202, 212.

In some embodiments, system 200 includes a processor (not illustrated) configured to receive signal/s from user input/s (e.g. one or more of input arm/s 206, 230, display 234, additional user interface/s 240, foot pedal 236). In some embodiments, the processor sends control signals to motor units 204, 218 and/or electrosurgical generator 224 e.g. based on signal/s received from user input/s.

In some embodiments, the processor sends control signals to control console actuator/s, for adjustment of portion/s of the control console. For example, in some embodiments, a user inputs a command through a user interface (e.g. display 234) to adjust one or more portion of the control console (e.g. position and/or orientation of input arm/s, height of a user support). In some embodiments, the processor generates a control signal, based on the user inputted command, the processor, for example, then sending the control signal to actuator/s to adjust the control console. In some embodiments, the processor generates the signal based on measurements, e.g. measured position/s and or movement/s of input arm/s. For example, in some embodiments, when input arms are moved (e.g. by a user) towards a collision, in some embodiments, a separation between the input arms is automatically increased, for example, by movement of one or more input arm support. Additionally or alternatively, an alert appears on the screen interface. Additionally or alternatively, surgical arm movement is halted.

In some embodiments, control console 228 includes a processor. Alternatively or additionally, in some embodiments, processing is hosted by an external processor which is, for example, configured to receive user input signal/s and/or send control signals to motor unit/s and/or the electrosurgical generator.

In some embodiments, foot pedal 236 and/or electrosurgical generator 224 include a processor configured to generate control signals (e.g. based on sensed pressure of user 232 pressing on portion/s of foot pedal 236). Where, for example, electrical power supplied to motor units is varied 208, 210 based on the control signals. In some embodiments, foot pedal control signals do not pass through a control console processor.

In some embodiments, a first input arm 204 controls movement of first surgical arm 202 and/or a second input arm 230 controls movement of second surgical arm 212. In some embodiments, a user positions and/or moves an input arm 206 by grasping an input arm handle 238.

In some embodiments, a system includes an electrosurgical switching unit, for example, connected between electrosurgical generator 212 and motor units 204, 218 which, for example, switches electrosurgical power supply (e.g. on and/or off) from the electrosurgical generator, for example, upon receiving a signal (e.g. from a electrosurgical switching unit user interface and/or from an external processor).

FIG. 18B is an example of an operating room setting, according to some embodiments.

In some embodiments, the surgical system 2200 comprises one or more surgical arms 2202 for insertion into the patient body. In some embodiments, surgical arms 2202 are actuated by a motor unit 2204. Optionally, the surgical arms are assembled on the motor unit by placing the arms through one or more locking doors 2210 of the motor unit.

In some embodiments, motor unit 2204 is positioned with respect to the surgical bed 2216 by support means such as a fixation arm 2206. In some embodiments, the fixation arm is constructed of a plurality of segments attached to each other by adjustable joints 2208. Optionally, joints are configured to be locked in position, for example by means of one or more lockable knobs (which, for example, is locked by rotation), latches, and/or other means suitable for adjusting and/or restricting a position of the fixation arm segments.

In some embodiments, electrosurgery cables 2212 for supplying monopolar or bipolar energy to the motor unit 2204 extend from an electrosurgical power generator 2214 to the motor unit.

In some embodiments, one or more cables 2218 extend from the motor unit to a control console 2220. In some embodiments, control console 2220 includes one or more input arms 2222 for controlling the one or more surgical arms 2202. In some embodiments, input arms 2222 are shaped and constructed to be controlled by a user's hand, finger (e.g. thumb), and/or part of the hand (e.g. controlled by gripping). In some embodiments, control console is mobile. Optionally, the control console is situated on a set of wheels 2224 which provide for pushing the console around. In some embodiments, control console 2220 includes user support, for example a seat 2226 and arm rests 2228. Optionally, seat 2226 is adjustable, for example in height, in a linear position with respect to the input arms, and/or in a lateral position with respect to the input arms. In some embodiments, seat 2226 is slidable along a base 2230 of the control console. Optionally, arm rests 2228 are adjustable, for example in height (e.g. relative to base 2230), and/or in their lateral position. In some embodiments, control console 2220 includes a display 2232, for example, for display of imaging during surgery (e.g. from a camera inserted with and/or mounted on surgical arm/s). Optionally, display 2232 is a touch screen and is configured to receive user inputs. In some embodiments, the console includes additional user interface/s, for example, in some embodiments including one or more switches or buttons 2234 such as an on/off switch, a light indicator, on/or off button (e.g. emergency off button), user interface/s on the input arm/s. In some embodiments, height and/or lateral position of the display is adjustable. In some embodiments, the control console comprises one or more storage compartments 2234, located for example behind display 2232.

In some embodiments, control console 2220 includes power and/or data connections 2236. Optionally, the control console comprises sockets for receiving electrical power from a mains supply. Additionally or alternatively, control console is powered by a battery.

FIG. 18C is a simplified schematic drawing of one or more surgical arms 2300, held by a support 2382 (e.g. a fixation arm), according to some embodiments.

In some embodiments, support 2382 attaches to a portion of a patient operating surface, e.g. to a rail 2302. In some embodiments, position of attachment of support 2382 on rail 2302 is adjustable, for example enabling linear adjustment of position of attachment of the support to the patient operating surface. Optionally, the adjustment is performed manually.

In some embodiments, support 2382 is attached to port 2312 of a motor unit 2314 (shown in this example without an external housing), surgical arms 2300 being supported by attachment to motor unit 2314.

In some embodiments, port 2312 is placed at an opening to the patient's body, for example at an incision and/or at a natural body orifice such as the vagina and/or anus and/or mouth. In some embodiments, port 2312 is attached to the patient's body using sutures and/or other attachment means. Additionally or alternatively, port 2312 is fixated to the operating surface bed.

In some embodiments, support 2382 includes a plurality of articulations where angles between segments and/or segment lengths are adjustable, for example, enabling adjustment of position and/or angle of the surgical arms and/or a port 2312 and/or motor unit 2314 (e.g. which actuate surgical arm/s 2300).

In some embodiments, one or more motor is used to move the surgical arms 2300, with respect to one or more portion of the system (e.g. with respect to port 2312 and/or motor unit 2314), for example, into and/or out of a patient. In some embodiments, motor unit 2314 includes one or more motor for movement of one or more surgical arms with respect to the motor unit, where, for example, one or more support segment position is changed with respect to the motor unit.

FIG. 18D is a simplified schematic drawing of a surgical mechanical arm controlled by an input arm, according to some embodiments.

In some embodiments, surgical mechanical arm 102 includes a first flexible portion 101 coupled to a second flexible portion 103, coupled to a tool 105 (e.g. an end-effecter, such as grasper, scissors, needle driver, driller, suction/irrigation tool, laser ablation tool and/or other tools.). In some embodiments, first flexible portion 101 defines a shoulder joint, and second flexible portion 103 defines an elbow joint. In some embodiments, a portion 109 defines a wrist joint.

In some embodiments, a distal portion of the surgical mechanical arm (e.g. including portions 101, 103, 105) is coupled to a surgical mechanical arm support 107. In some embodiments, support 107 is rigid.

In some embodiments, surgical arm 102 is actuated by a motor unit 104. In some embodiments, surgical mechanical arm 102 is supplied with electrical power e.g. for electrosurgery through motor unit 104. In some embodiments, motor unit 104 receives electrosurgical power from an electrosurgical power generator (not illustrated).

In some embodiments, input arm 106 includes a first flexion joint 150 and a second flexion joint 152. In some embodiments, one or more sensors 108 in communication with the surgical arm and the input arm senses a position of one or more portion of input arm 106. In some embodiments, sensor/s 108 measure movement between portion/s of the input arm, for example, flexion at flexion joints 150. 150 and/or rotation about rotational joint/s 160, 162, 174.

In some embodiments, a processor 110 receives a signal from sensor/s 108 and generates one or more control signal. In some embodiments, processor 110 sends the generated control signal to motor unit 104 which, in some embodiments, actuates movement of surgical mechanical arm 102, based on the control signal.

In some embodiments, the processor instructs the motor unit to move the surgical mechanical arm into a configuration where a shape of the surgical arm corresponds to a shape of the input arm. For example, where the surgical device has about the same angles between corresponding segments as the input device. Where, in some embodiments, angles between segments are measured as intersections between central long axes of the arm at rotational joints.

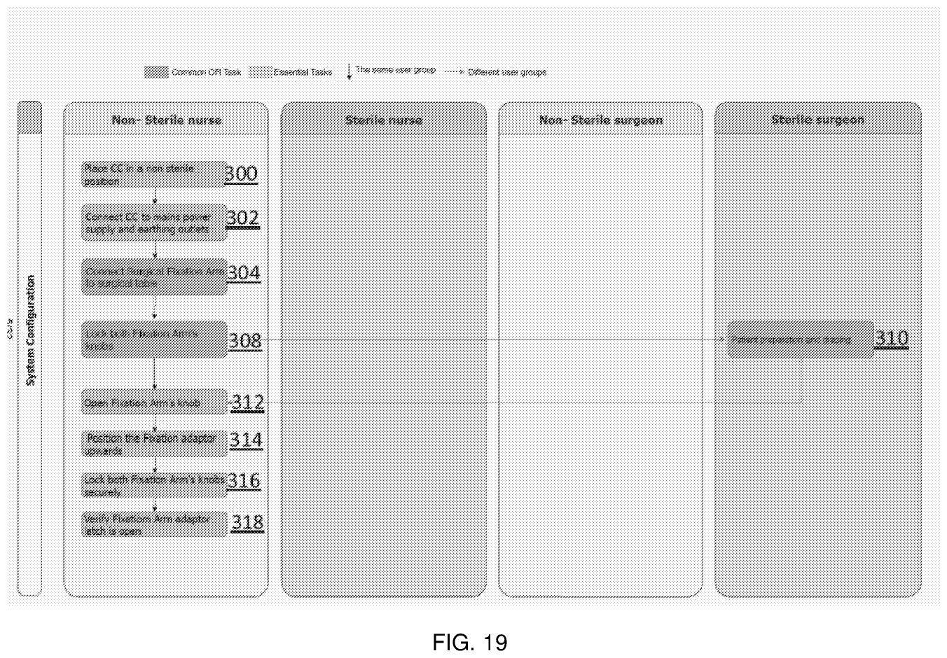

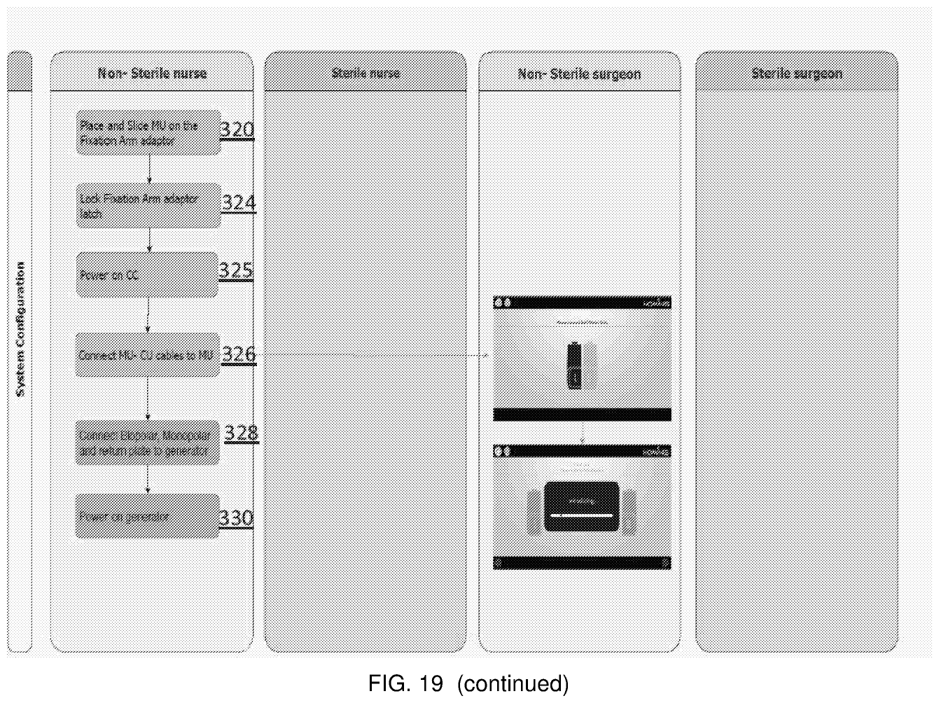

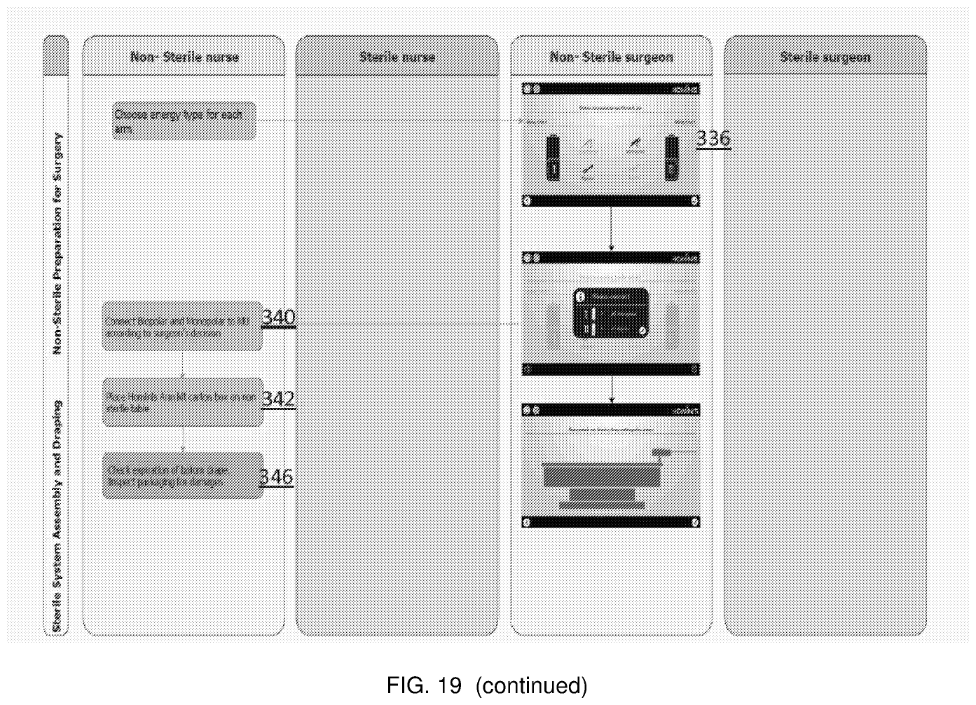

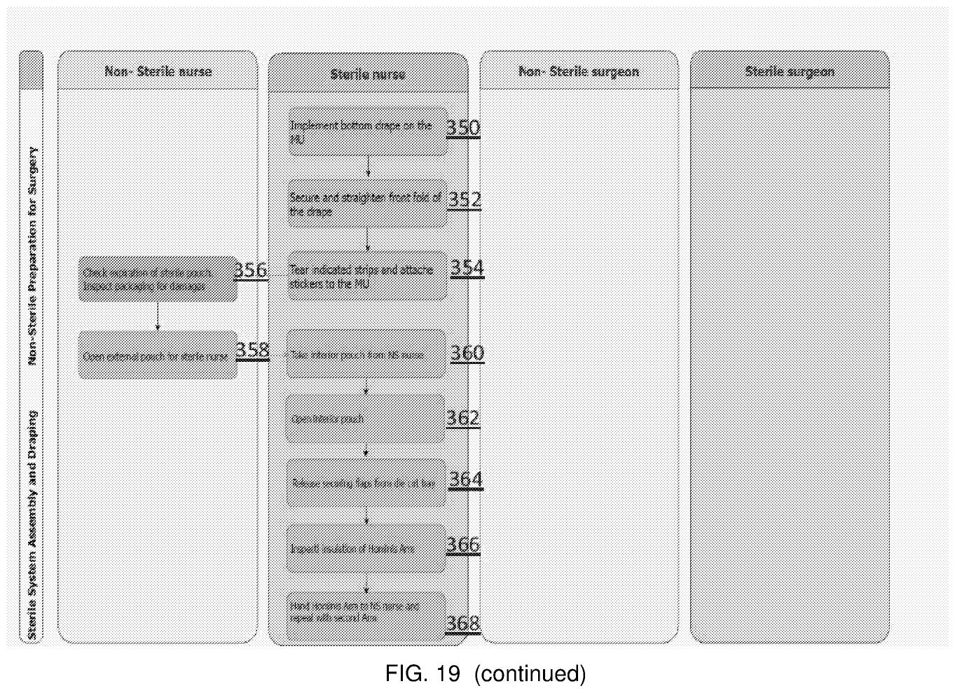

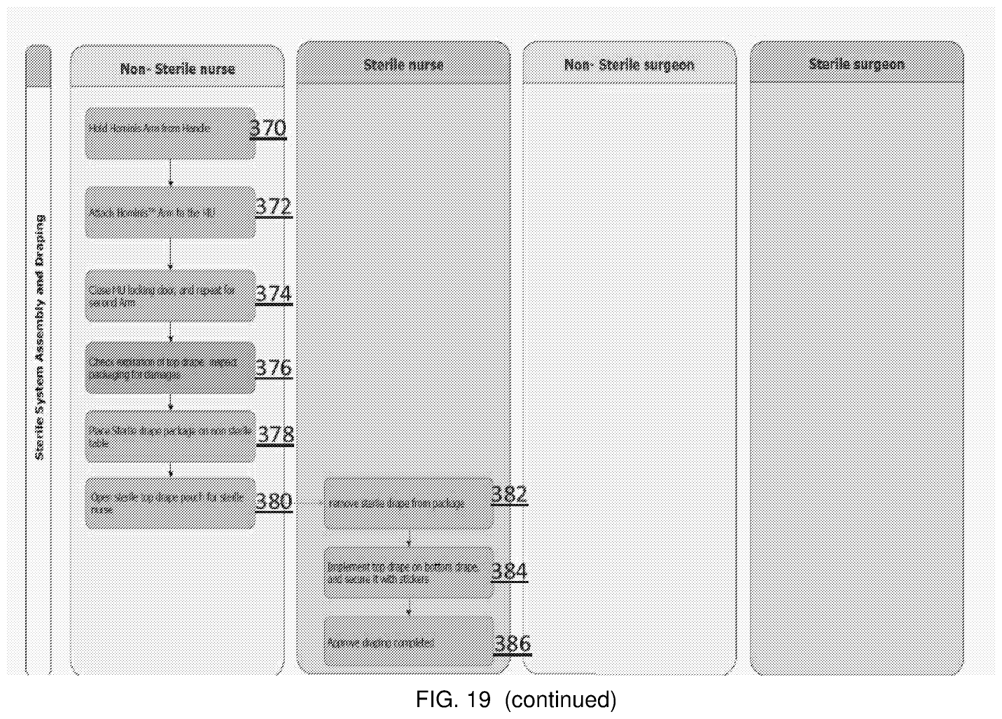

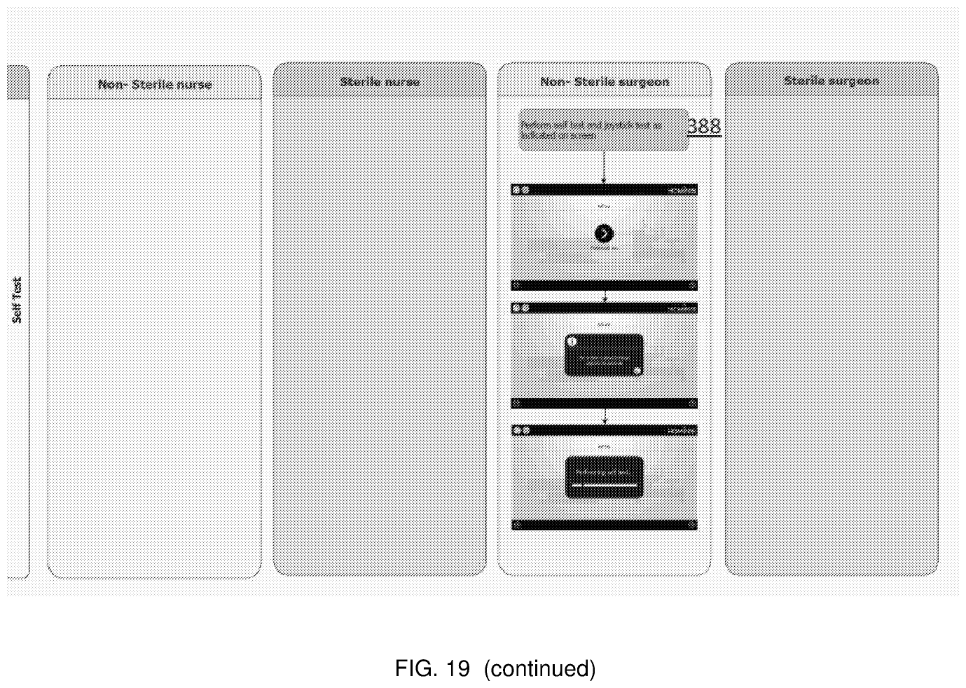

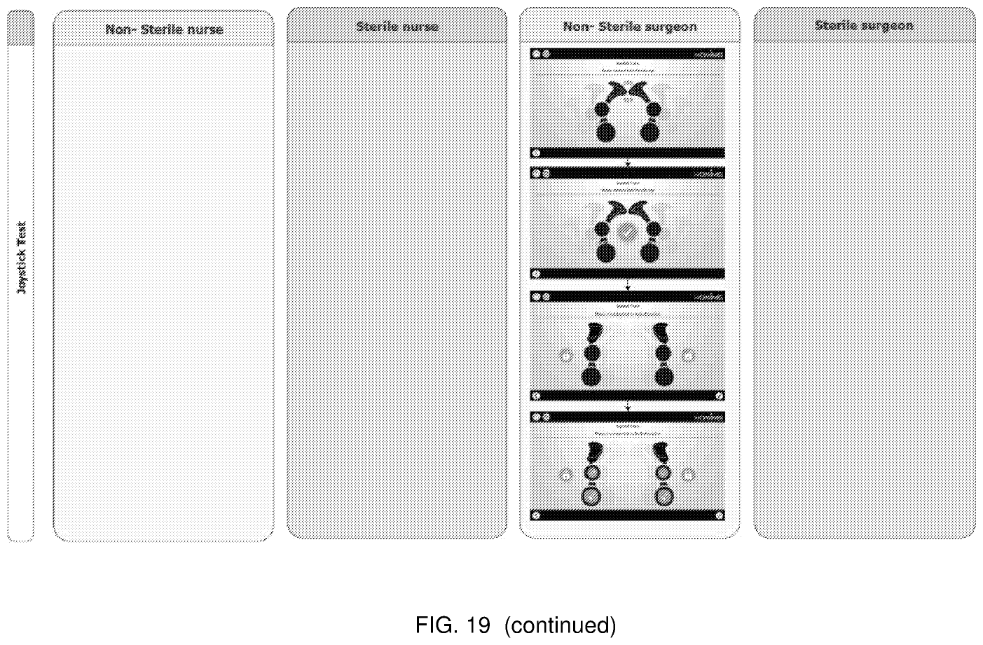

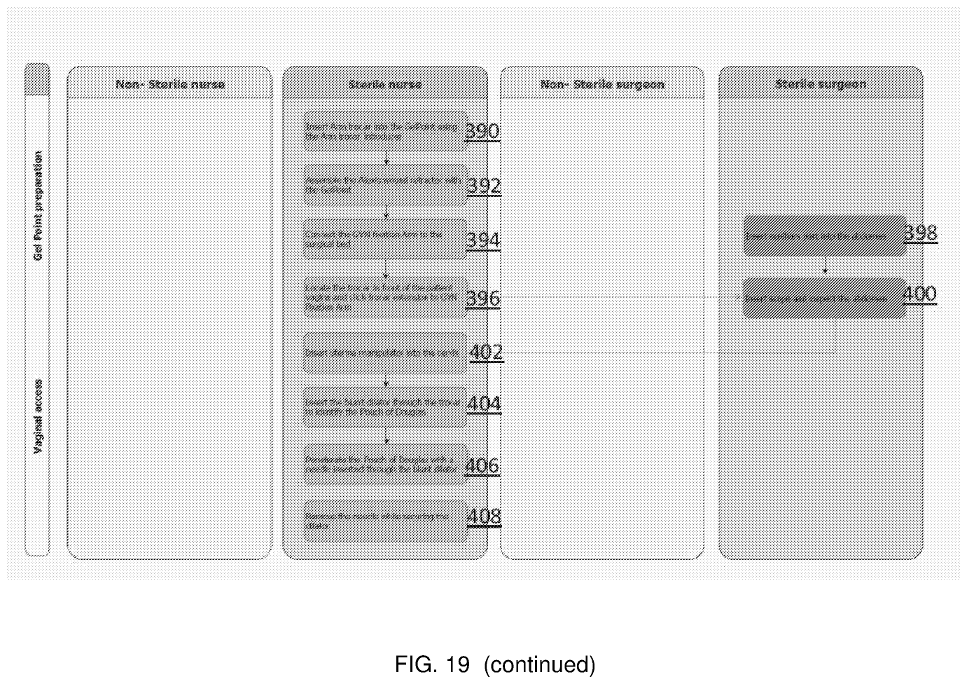

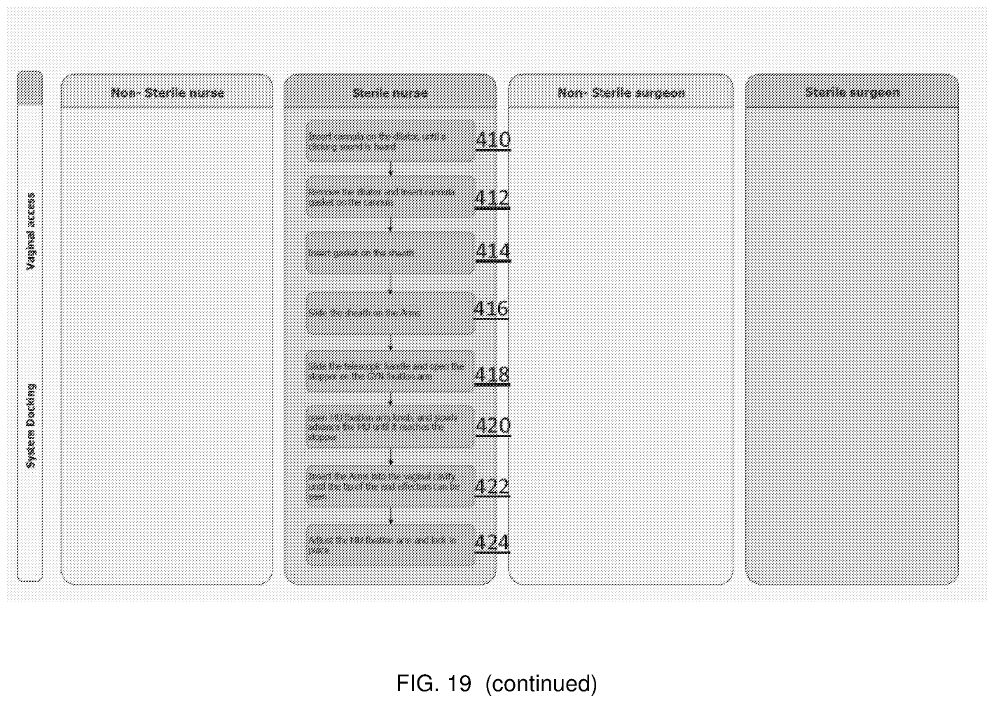

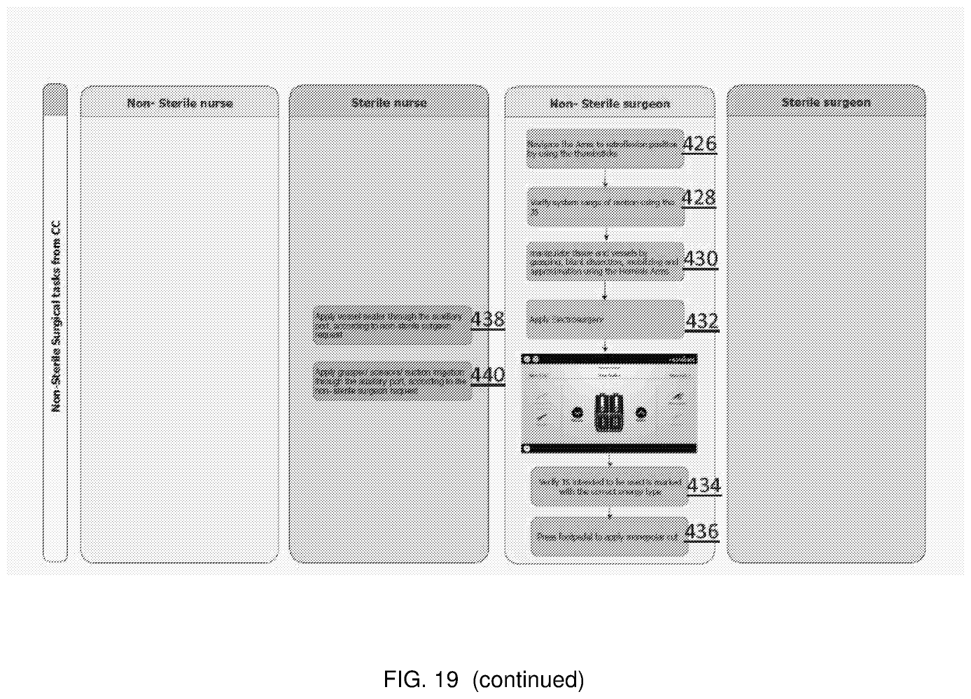

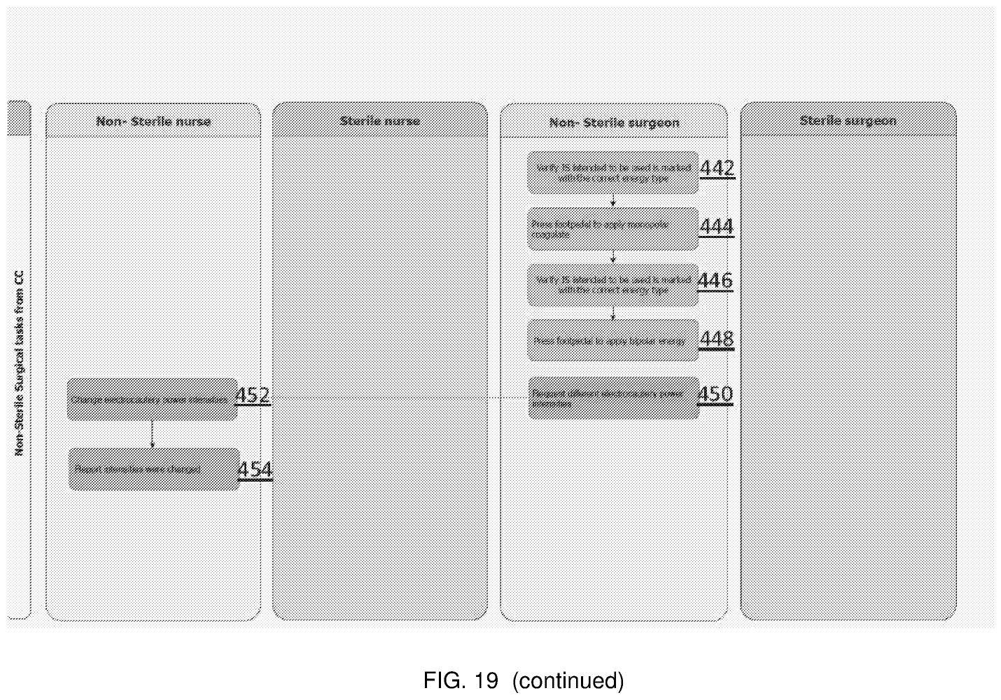

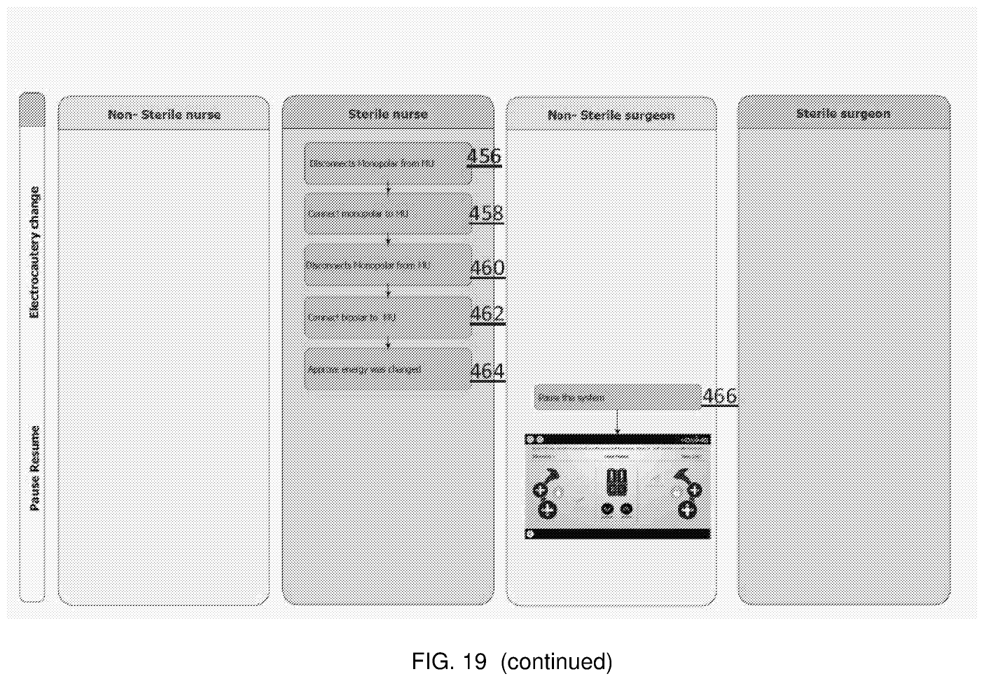

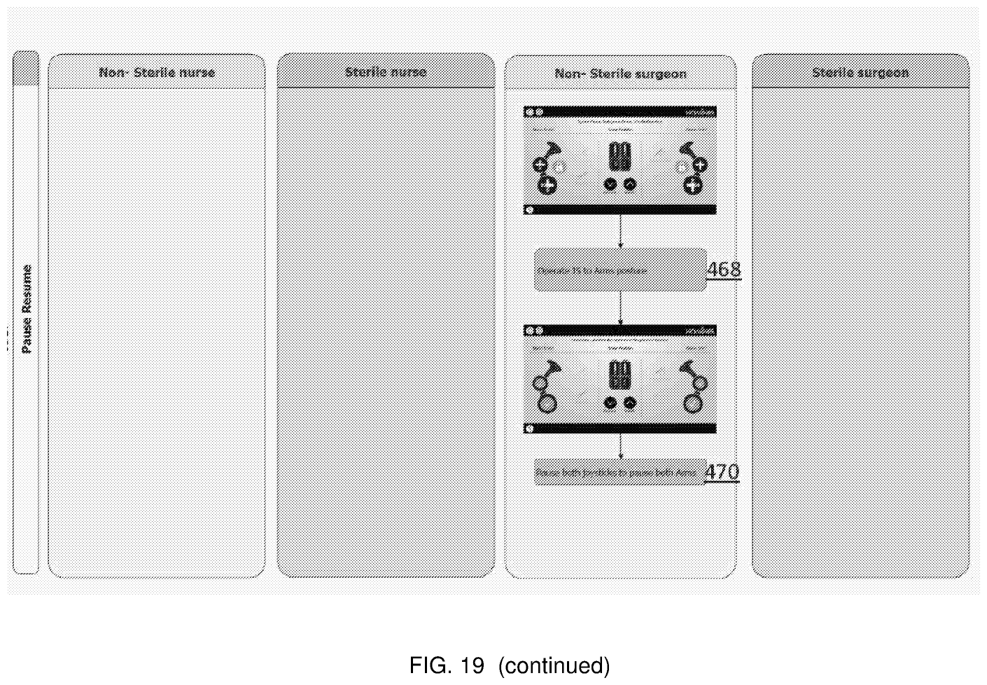

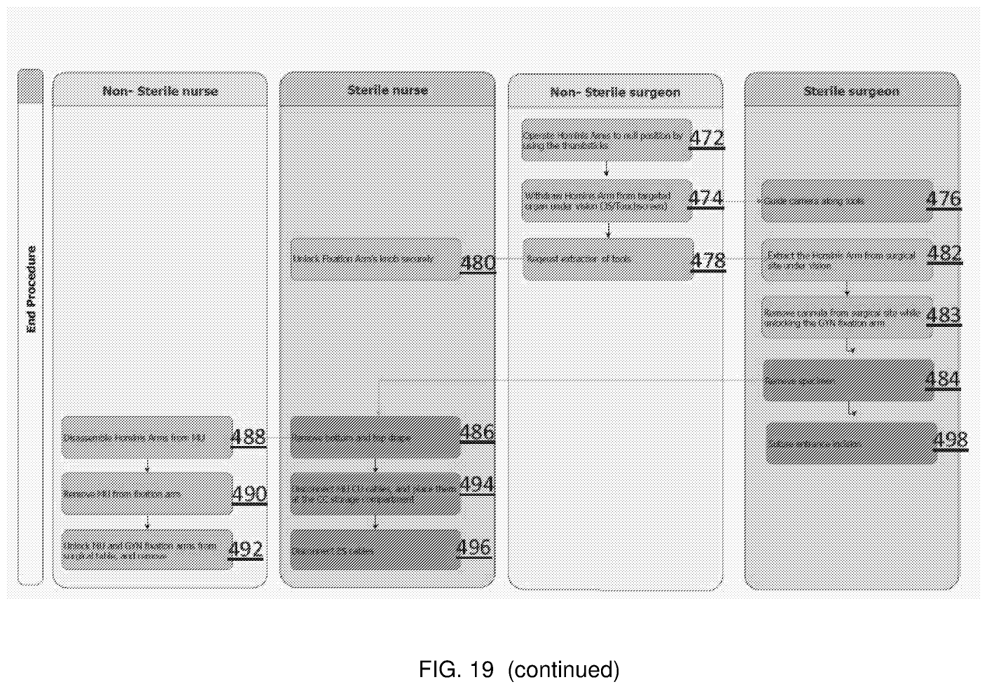

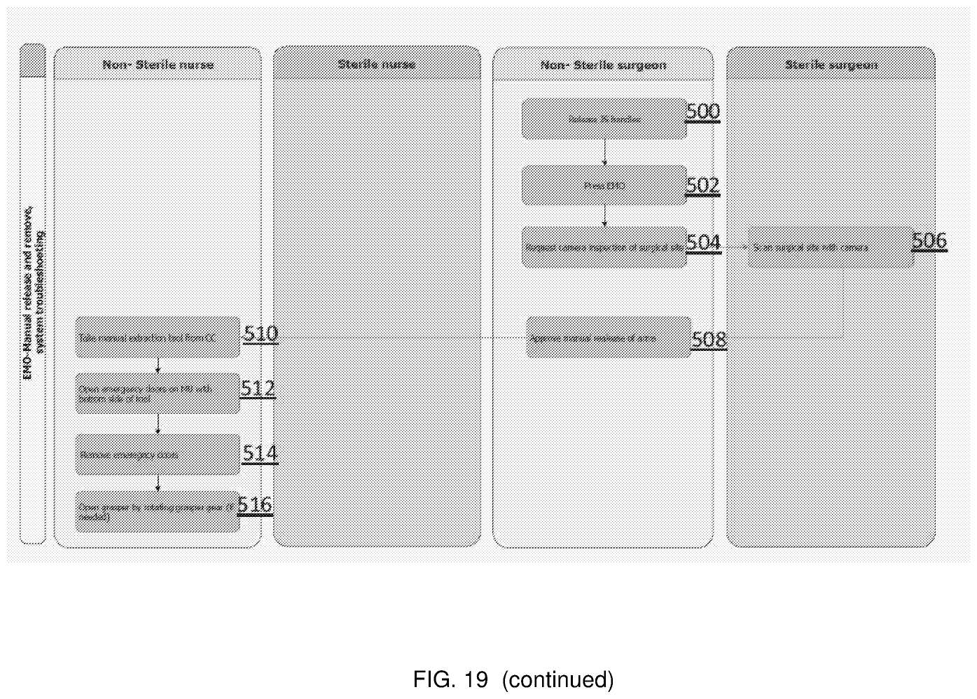

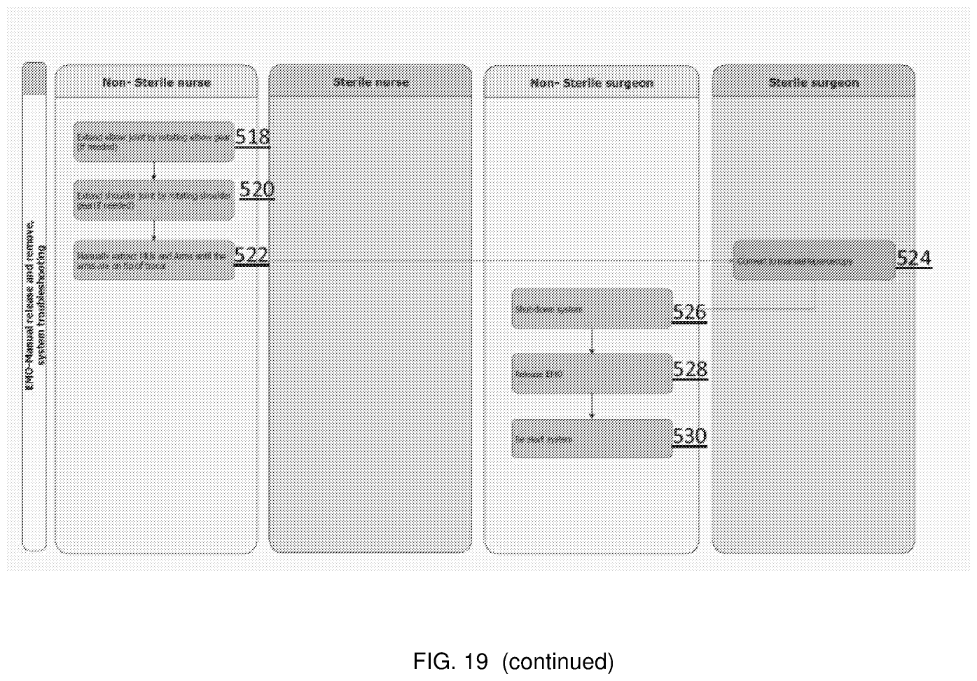

FIG. 19 is a flow chart of a detailed use flow of a medical robotic surgical system, according to some embodiments.

### System Configuration

At 300, in some embodiments, a non-sterile nurse places a control console (CC) in a non-sterile position.

At 302, in some embodiments, the non-sterile nurse connects the control console to a mains power supply and earthing outlets.

At 304, in some embodiments, the non-sterile nurse connects the surgical fixation arm to a surgical bed.

At 308, in some embodiments, the non-sterile nurse locks one or more fixation arm knob e.g. both knobs of two knobs.

At 310, in some embodiments, the sterile surgeon prepares the patient including, for example, draping the patient.

At 312, in some embodiments, the non-sterile nurse opens one or more surgical fixation arm knob.

At 314, in some embodiments, the non-sterile nurse positions the fixation arm adaptor upwards.

At 316, in some embodiments, the non-sterile nurse re-locks one or more fixation arm knob e.g. both knobs of two knobs.

At 318, in some embodiments, the non-sterile nurse verifies that a fixation arm adaptor latch is open.

At 320, in some embodiments, the non-sterile nurse places and slides a motor unit (MU) (optionally, a part of the motor unit, such as a dove tail of the motor unit housing) into the fixation arm adaptor.

At 324, in some embodiments, the non-sterile nurse locks the fixation arm adaptor latch.

At 325, in some embodiments, the non-sterile nurse turns on the control console.

At 326, in some embodiments, the non-sterile nurse connects motor unit-control unit (MU-CU) cables to the motor unit.

At 328, in some embodiments, the non-sterile nurse connects bipolar and/or monopolar cables from the electrosurgical generator to the control console and optionally (e.g. in the case where monopolar electrosurgery is to be employed) the non-sterile nurse connects a return plate to the electrosurgical generator.

At 330, in some embodiments, the non-sterile nurse turns (e.g. electrically activates e.g. using a user interface) on the electrosurgical generator.

### Preparation for Surgery, System Assembly and Draping

At 336, in some embodiments, the non-sterile surgeon selects an electrosurgical power type, for example, for each surgical arm for example, from a selection of electrosurgical power types displayed at the control console user interface (e.g. at GUI screen display).

At 340, in some embodiments, the non-sterile nurse connects bipolar and/or monopolar cables to one or more motor unit according to the surgeon's selection at step 336.

At 342, in some embodiments, the non-sterile nurse places a surgical arm kit carton box on a non-sterile table.

At 346, in some embodiments, the non-sterile nurse checks an expiration date of a surgical drape for covering the motor unit (for example, a bottom drape sheet when top and bottom drape sheets are used) and/or inspects packaging for damage.

At 350, in some embodiments, a sterile nurse implements the bottom drape on the motor unit.

At 352, in some embodiments, the sterile nurse secures and straightens a front fold of the bottom drape.

At 354, in some embodiments, the sterile nurse tears one or more indication strips and attaches one or more adhesives (e.g. stickers) to attach the drape to the motor unit.

At 356, in some embodiments, the non-sterile nurse checks an expiration date of the sterile pouch (optionally a double layered sterile pouch) and/or inspects packaging for damage.

At 358, in some embodiments, the non-sterile nurse opens an external pouch of the double-layered pouch.

At 360, the sterile nurse takes an interior pouch from the double-layered pouch from the non-sterile nurse.

At 362, in some embodiments, the sterile nurse opens interior the interior pouch.

At 364, in some embodiments, the sterile nurse releases one or more securing flaps from a die cut tray or releases one on more inserts from a blister in which the surgical mechanical arm is packed. (In some embodiments, the surgical arms are packed in a die cut tray or a blister for sterilization purposes performed prior to the surgery).

At 366, in some embodiments, the sterile nurse inspects insulation of surgical mechanical arm.

At 368, in some embodiments, the sterile nurse hands the surgical mechanical arm to the non-sterile nurse, while holding a sterile end of the arm. Optionally, the process is repeated with a second surgical mechanical arm.

At 370, in some embodiments, the non-sterile nurse holds the surgical mechanical arm at a handle of the arm.

At 372, in some embodiments, the non-sterile nurse attaches surgical mechanical arm to the motor unit.

At 374, in some embodiments, the non-sterile nurse closes motor unit locking door. Optionally, the process is repeated with a second surgical mechanical arm.

At 376, in some embodiments, the non-sterile nurse checks an expiration date of the top surgical drape, and optionally inspects packaging for damages.

At 378, in some embodiments, the non-sterile nurse places the drape package on the non-sterile table.

At 380, in some embodiments, the non-sterile nurse opens the top drape pouch for the sterile nurse.

At 382, in some embodiments, the sterile nurse removes the drape from the package;

At 384, in some embodiments, the sterile nurse implements the top drape on the bottom drape, and secures the top drape with adhesives (e.g. stickers).

At 386, in some embodiments, the sterile nurse approves that draping is completed.

### Self-Test and Input Arm Test

At 388, in some embodiments, the non-sterile surgeon performs a self-test and/or an input arm (e.g. joystick) test according to instructions on the GUI screen. During the self test, in some embodiments, the surgeon operates the input arms according to displayed instructions (for example: contacts tips of input arm handles against each other, moves each of the input arms to a locked position and/or to an unlocked position, and the like).

### Port Preparation, Vaginal Access and System Docking

At 390, in some embodiments, the sterile nurse inserts an arm trocar (for example, a designated trocar shaped and sized for insertion of the one more surgical arms through) into a surgical port (optionally comprising a “GelPoint” port) using an arm trocar introducer.

At 392, in some embodiments, the sterile nurse assembles a wound retractor (such as “Alexis” wound retractor) with the surgical port.

At 394, in some embodiments, the sterile nurse connects the GYN fixation arm to the surgical bed.

At 396, in some embodiments, the sterile nurse locates the arm trocar in front of the patient's vagina and attaches an extension of the trocar to the GYN fixation arm (optionally by clicking).

At 398, in some embodiments, the sterile surgeon inserts an auxiliary port into the patient's abdomen.

At 400, in some embodiments, the sterile surgeon inserts a scope and inspects the abdomen;

At 402, in some embodiments, the sterile nurse or the sterile surgeon inserts a uterine manipulator into the cervix.

At 404, in some embodiments, the sterile nurse or the sterile surgeon inserts a blunt dilator through the trocar to identify the Pouch of Douglas.

At 406, in some embodiments, the sterile nurse or the sterile surgeon inserts a needle through the blunt dilator and penetrate the Pouch of Douglas.

At 408, in some embodiments, the sterile nurse or the sterile surgeon removes the needle while securing the dilator.

At 410, in some embodiments, the sterile nurse inserts a cannula on the dilator, until a “click” sound indication is heard.

At 412, in some embodiments, the sterile nurse removes the dilator and places a cannula gasket on the cannula.

At 414, in some embodiments, the sterile nurse inserts the gasket on a sheath.

At 416, in some embodiments, the sterile nurse slides the sheath on the surgical arms.

At 418, in some embodiments, the sterile nurse or the sterile surgeon slides a telescopic handle and opens a stopper on the GYN fixation arm. At 420, in some embodiments, the sterile nurse opens the motor unit fixation arm knob, and slowly advances the motor unit until it reaches the stopper on the GYN fixation arm.

At 422, in some embodiments, the sterile nurse inserts the surgical arms into the vaginal cavity, until the tip of the end effecters can be seen, for example on a screen such as one that shows a video captured by the laparoscopic camera. At 424, in some embodiments, the sterile nurse adjusts the motor unit fixation arm and locks it in place.

### Non-Sterile Surgical Tasks Performed Via Control Console

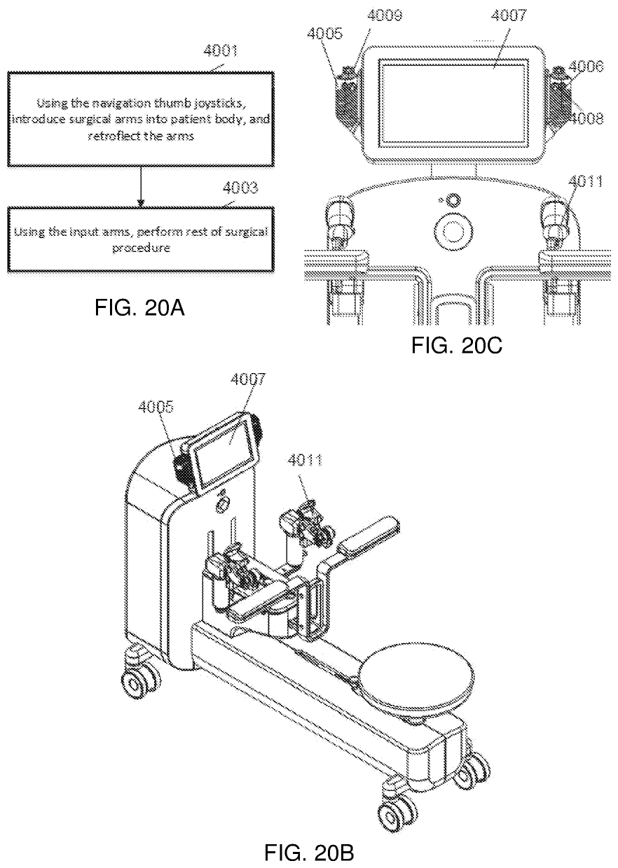

At 426, in some embodiments, the non-sterile surgeon navigates the surgical arms to a retroflexed position, optionally using a set of thumbsticks (for example as described in FIGS. 20A-C).

At 428, in some embodiments, the non-sterile surgeon verifies the range of motion of the system using the input arms (e.g. joysticks).

At 430, in some embodiments, the non-sterile surgeon manipulates tissue and vasculature using one or more of grasping, blunt dissecting, mobilizing and/or approximating tissue using the surgical arms.

At 432, in some embodiments, the non-sterile surgeon applies electrosurgery, optionally via the GUI screen.

At 434, in some embodiments, the non-sterile surgeon verifies that the input arm (e.g. joystick) intended to be used is marked with the correct energy type.

At 436, in some embodiments, the non-sterile surgeon presses the foot pedal to apply a monopolar cut.

At 438, in some embodiments, the sterile nurse or sterile surgeon applies a vessel sealer through the auxiliary port, in response to a request from the non-sterile surgeon. In some embodiments, sealing is by applying energy such as bipolar energy to the vessel to coagulate the tissue. In some embodiments, a dedicated instrument (e.g. scissors, a curved tool, and/or others) configured for sensing the tissue reaction to the applying of energy, and optionally modifying the applied current according to the sensed tissue characteristics is used for sealing. At 440, in some embodiments, the sterile nurse or sterile surgeon introduces a grasper, scissors, suction and/or irrigation through the auxiliary port, in response to a request from the non-sterile surgeon.

At 442, in some embodiments, the non-sterile surgeon verifies that the input arms (e.g. joystick) intended to be used is marked with the correct energy type.

At 444, in some embodiments, the non-sterile surgeon presses the foot pedal to apply monopolar coagulation.

At 446, in some embodiments, the non-sterile surgeon verifies that the input arms (e.g. joystick) intended to be used is marked with the correct energy type.

At 448, in some embodiments, the non-sterile surgeon presses the foot pedal to apply bipolar energy.

At 450, in some embodiments, the non-sterile surgeon requests different electrocautery power intensities.

At 452, in some embodiments, the non-sterile nurse changes the electrocautery power intensities in response to the request of the non-sterile surgeon.

At 454, in some embodiments, the non-sterile nurse reports that the intensities were changed.

### Electrocautery Change

At 456, in some embodiments, the sterile nurse disconnects the monopolar energy cable from the motor unit.

At 458, in some embodiments, the sterile nurse connects the monopolar energy cable to the motor unit.

At 460, in some embodiments, the sterile nurse disconnects the monopolar energy cable from the motor unit.

At 462, in some embodiments, the sterile nurse connects the bipolar energy to the motor unit.

At 464, in some embodiments, the sterile nurse approves that the energy type was changed.

At 466, in some embodiments, the non-sterile surgeon pauses the system and follows the instructions on the GUI screen (for example, moving the input arms (e.g. joysticks) to a locked position before taking his hands off).

### Pause and Resume

At 468, in some embodiments, the non-sterile surgeon resumes the system by following instructions on the GUI screen (for example, operating the input arms according to an indicated posture).

### End of Procedure

At 472, in some embodiments, the non-sterile surgeon operates the surgical arms to a null position, optionally using the thumbsticks.

At 474, in some embodiments, the non-sterile surgeon withdraws the surgical arms from the target organ under vision (for example using the input arms and/or touchscreen).

At 476, in some embodiments, the sterile surgeon guides a camera along one or more tools that were previously inserted, for example during extraction of the tools. Optionally, the tool is straightened during extraction. Optionally, the camera provides a complete view of the surgical arm, including the tool (e.g. from a proximal end of the surgical arm to a distal end of the tool). In some embodiments, the camera focus is zoomed out to obtain the complete view of the surgical arm, for example to ensure that arm joints such as the elbow and/or shoulder joints do not contact organs during extraction.

At 478, in some embodiments, the non-sterile surgeon requests extraction of tools.

At 480, in some embodiments, the sterile nurse unlocks the fixation arm knob securely.

At 482, in some embodiments, the sterile surgeon extracts the surgical arm from the surgical site under vision;

At 483, in some embodiments, the sterile surgeon removes the cannula from the surgical site while unlocking the GYN fixation arm.

At 484, in some embodiments, the sterile surgeon removes a specimen from the surgical site. Optionally, the specimen comprises an organ that was removed, such as uterus, ovaries, fallopian tubes, gallbladder, and/or other organs or portions thereof. In some embodiments, target tissue (e.g. a tumor) is removed.

At 486, in some embodiments, the sterile nurse removes the top and bottom drapes.

At 488, in some embodiments, the non-sterile nurse disassembles the surgical arms from the motor unit.

At 490, in some embodiments, the non-sterile nurse removes the motor unit from the fixation arm.

At 492, in some embodiments, the non-sterile nurse unlocks the motor unit and GYN fixation arms form the surgical bed, and removes them.

At 494, in some embodiments, the sterile nurse disconnects the motor unit-control unit cables, and places the cables at the control console storage compartment.

At 496, in some embodiments, the sterile nurse disconnects the electrosurgery cables.

At 498, in some embodiments, the sterile surgeon applies stitches to the entrance incision. In some embodiments, for example if a hysterectomy is performed, stitches are applied to suture together the vaginal cuff.

### Emergency Manual Release, System Troubleshooting

At 500, in some embodiments, the non-sterile surgeon releases the input arm (e.g. joystick) handles.

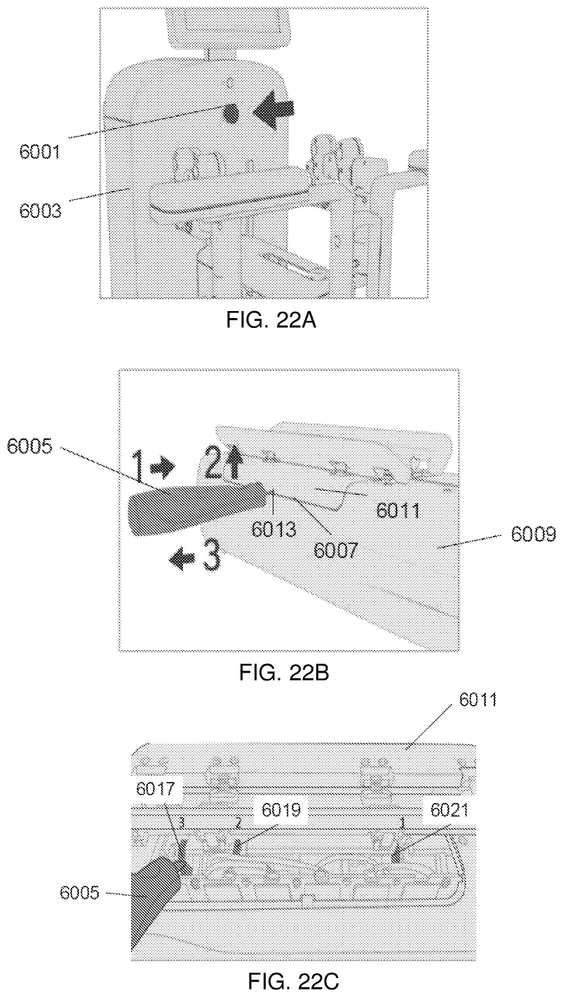

At 502, in some embodiments, the non-sterile surgeon presses the emergency button (EMO), see for example FIG. 22A.

At 504, in some embodiments, the non-sterile surgeon requests camera inspection of the surgical site.

At 506, in some embodiments, the sterile surgeon scans the surgical site with a camera.

At 508, in some embodiments, the non-sterile surgeon approves manual release of the surgical arms.

At 510, in some embodiments, the non-sterile nurse picks up a manual extraction tool from the control console. An example of a manual extraction tool is described in FIGS. 22B-C below.

At 512, in some embodiments, the non-sterile nurse opens emergency doors configured on the motor unit with the tool (optionally with a bottom portion of the tool).

At 514, in some embodiments, the non-sterile nurse or non-sterile surgeon removes the emergency doors.

At 516, in some embodiments, if required, the non-sterile nurse opens the end-effecter (such as a grasper, for example by rotating the grasper gear).

At 518, in some embodiments, if required, the non-sterile nurse extends the elbow joint of the surgical arm by rotating the elbow gear.

At 520, in some embodiments, if required, the non-sterile nurse extends the shoulder joint of the surgical arm by rotating the shoulder gear.

At 522, in some embodiments, the non-sterile nurse manually extracts the motor unit and surgical arms until the arms are positioned at the tip of the trocar.

At 524, in some embodiments, the sterile surgeon converts to manual laparoscopy, optionally aborting the robotic surgical and continuing with traditional abdominal laparoscopy.

At 526, in some embodiments, the non-sterile surgeon shuts down the system.

At 528, in some embodiments, the non-sterile surgeon releases the emergency button.

At 530, in some embodiments, the non-sterile surgeon re-starts the system.

### Control of Mechanical Surgical Arms

FIGS. 20A-C are a flowchart of a method for dual-control of surgical arms (FIG. 20A) and schematic drawings of a control console comprising dual control means (FIGS. 20A-B), according to some embodiments.

In some embodiments, control of the one or more surgical arms is achieved via one or more input arms, joysticks, control handles, and/or other means suitable for manipulation by a user (e.g. a surgeon) which is then translated into a matching articulation of the surgical arm(s).

In the example described herein, as referred to in the flowchart of FIG. 20A, some embodiments include dual-control of the surgical arms. In some embodiments, a first user input (in this example, thumbsticks 4005) is used for introducing the surgical arms into the patient body, for example through the vagina, and then for retroflecting the surgical arms (4001). In some embodiments, retroflecting (for example, bending backwards) of the surgical arms within the patient body is performed to reduce an area within which the surgical arm is located. Optionally, retroflecting is performed during a surgical procedure to avoid obstacles such as certain organs or portions thereof, for example, an inner wall of the abdomen. Optionally, retroflecting is performed to position the surgical arms at an orientation in which a laparoscopic surgeon is familiar with, for carrying out the operation.

In some embodiments, a second user input, in this example in the form of input arms 4011 (e.g. avatar joysticks) is then used for performing the rest of the surgical procedure (4003).

In some embodiments, thumbsticks 4005 (see FIGS. 20B-C) are positioned adjacent the control console screen 4007, for example on opposing sides of the screen. In some embodiments, each of the thumbsticks 4005 comprises a nipple type controller 4009 shaped and sized for the user's thumb. In some embodiments, the nipple of the thumbsticks is at rest position when centered, and is configured to spring back to the rest position upon release of the thumb. In some embodiments, the extent of movement of the nipple relative to its central rest position determines a resulting velocity of the surgical arm movement. For example, the further the nipple is pushed away from its central rest position, the higher the velocity of the movement of the surgical arm (and vice versa—the closer the nipple is to its central rest position, the lower the velocity of the arm).

In some embodiments, when controlling the surgical arms via the thumbsticks, one or more of the surgical arm joints (e.g. a shoulder joint, a wrist joint) are restricted from movement. In some embodiments, all surgical arm joints except an elbow joint are prevented from moving, and only flexion and/or rotation of the elbow joint are enabled. In some embodiments, linear movement of the surgical arm (as a single body) is also enabled, for example to advance or retract the arm. In some embodiments, movement of the nipple actuates flexion and/or rotation of the elbow joint. In some embodiments, linear movement of the arm is actuated by separate actuators, for example using push buttons such as 4006, 4008, configured for example along a body of the thumbstick 4005. In an example, button 4006 advances the surgical arm distally (e.g. into the abdomen); button 4008 retracts the surgical arm proximally.

In some embodiments, during use of the thumbsticks, the input arms 4011 are locked at a rest position, for example by solenoid locks. In some embodiments, the rest position of the input arms is selected as the retroflected position. Optionally, this position allows the surgeon to continue the procedure directly following retroflection using the thumbsticks. In some embodiments, when the input arms are operated, operation of the thumbsticks is disabled.

A potential advantage of using the thumbsticks for navigation into the body and for retroflecting the surgical arms while selected arm joints such as the shoulder joint remain stationary may include reducing a bending radius of the surgical arm, thereby reducing a likelihood of encountering surrounding obstacles such as the inner abdominal wall. Another potential advantage of using the thumbsticks for navigation and/or retroflection processes may include improved control over the surgical arms, for example as compared to navigating and retroflecting with the input arms in which the ergonomics of the handle may be less suitable for supporting the rotational movement that the surgeon needs to perform while holding the handle in order to carry out retroflection.

In some embodiments, during introducing of the surgical arms into the body the surgical arms are straight (optionally to provide for insertion via a cannula), while the input arms are at a rest, locked, retroflected position. Optionally, following retroflection of the surgical arms using the thumbsticks, the surgeon releases the thumbsticks and moves their hands to the input arms. Once the input arms are grasped and optionally lifted by the surgeon, control over the surgical arms is automatically gained, and the surgeon may continue the procedure using the input arms. In some embodiments, when one or more input arm joints are locked by solenoid locks, lifting of the input arm by the surgeon automatically releases the solenoid locks. Additionally or alternately, manual locks of the input arm joints are released, for example via a sensor that detects an input arm position.

In some embodiments, the system (e.g. a system processor) is configured to recognize one or more positons of the input arms, for example when the input arms are at their rest position, and optionally display to the current position to the user.

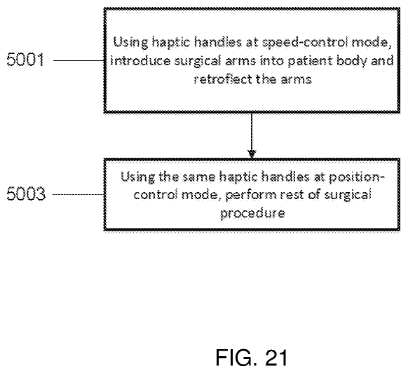

FIG. 21 is a flowchart of a method of using haptic handles to control one or more surgical arms, according to some embodiments.

In some embodiments, haptic handles which provide force feedback to the user (such as “omega.7” by “force dimension”) are used throughout the operation for controlling movement and articulation of the surgical arms. In some embodiments, the haptic handles are set to provide counter resistance for preventing the user from moving in directions that are not supported by the surgical arm, for example bending the elbow joint of the surgical arm backwards; touching a joint (e.g. an elbow joint) with a different segment of the same arm; and/or other. In some embodiments, the handles are set to provide counter resistance which varies in accordance with a current anatomical position and/or orientation of the surgical arms. In an example, resistance may be increased if the user attempts a disallowed anatomical area, such as an organ that should be avoided.

In some embodiments, the haptic handles are programmed to operate according to various control modes. Optionally, the control mode is selected in accordance with a current stage in the surgical operation. In some embodiments, switching between different modes is performed via one or more of a screen interface, one or more buttons on the control console or handles, a foot pedal, and/or other.

In some embodiments, during a first stage of the procedure, during which the surgical arms are introduced into the patient body and optionally retroflected, the haptic handles are used in “speed-control” mode (5001). Optionally, in the speed-control mode, relative movement of a handle with respect to the handle rest position sets the speed in which the surgical arm is moved. As the user moves the handle further away from the rest position, the speed is raised, and vice versa. For example, movement of the handle to the right of its rest position may result in rotation of an arm joint (e.g. elbow joint) to the right, at a speed determined according to the distance of the handle from its rest position. In some embodiments, in the speed-control mode, the haptic handles are set to provide an elastic (spring-like) counter resistance to the movement of the user. In some embodiments, in the speed control mode, a control algorithm is applied, converting a current configuration of the haptic handle into speed commands issued to the actuators (e.g. motors) of the surgical arm, such as to increase a rotation speed of one or more motor gears.

A potential advantage of using the speed control mode during introducing and optionally retroflecting the surgical arms in the body may include that during retroflecting the directions are reversed (e.g. upwards/downwards), yet that change can be ignored and the motion can be naturally continued, since the resulting movement of the surgical arms is limited and what changes is the speed of movement.

In some embodiments, during a second stage of the surgical procedure, optionally during the rest of the procedure, the haptic handles are set to “position-control” mode (5003). Optionally, in the position control mode, a spatial position of the handles sets a respective position of the surgical arms. In the position control mode, a user's displacement of the haptic handle is translated to a relative displacement command to the surgical arm. In some embodiments, converting the displacement of the haptic handle is controlled according to an algorithm. In some embodiments, control is according to known algorithms (e.g. the Inverse Jacobian algorithm). Additionally or alternatively, in some embodiments, control is according to custom algorithms. In an example, a custom algorithm is set to scale the user's motion, such as to increase accuracy of movement. Such scaling may include amplifying the movement required on the user end by a selected coefficient to produce a similar non-amplified movement of the surgical arm. For example, for the arm to move a distance X, the user will need to move the handle by A*X (A>1). In another example, an algorithm is selected to filter signals, for example filtering using a low-pass filter to reduce user hand tremors.

In some embodiments, in the position control mode, a clutching mechanism is provided, allowing a user to temporarily disconnect from the surgical arm (such that movement of the input haptic handle no longer controls the surgical arm). Optionally, when disconnected, the user may freely re-position the haptic handle. In an example, the user re-positions the haptic handle to a position and/or orientation in which it is more comfortable for the user to carry out and control the next movement.

In some embodiments, the extent of resistance sensed by the user in response to movement of the handles is selected and controlled. In an example, a floating mode is set, in which the user substantially does not encounter resistance and is free to move the handle in all directions. Additionally or alternatively, a level of resistance sensed by the user is adjusted, for example so that the user senses a high resistance in response to one movement and a low or no resistance in response to another movement.

In some embodiments, the amount of resistance is controlled based on the anatomical location of the surgical arms. For example, a high resistance may be set where obstacles (e.g. the abdominal wall) are found near the surgical arm. In a specific example, if an obstacle is found on the right of the surgical arm, the user may encounter high resistance in response to moving the handle to the right; if no obstacles are found on the left of the arm, the user may encounter low or no resistance in response to moving the handle to the left. Optionally, the extent of resistance is defined by setting system definitions such as producing wall type resistance, rubber like resistance, sand type resistance and/or other.

### Emergency Release Method and Tools

FIGS. 22A-C illustrate stages and tools used during an emergency release of the surgical arms, according to some embodiments.

FIG. 22A shows an example of an emergency button 6001 configured on a control console 6003. In some embodiments, in case of an emergency, such as device malfunction, patient emergency, a problem at the user (surgeon) end, and/or hospital power shut down, button 6001 is pushed by a user to stop movement of the surgical arms. In some embodiments, upon pushing the button, the surgical arms remain static at the last position the arms were in. Alternatively, in some embodiments, the arms are moved to a rest position (optionally a predefined rest position), including for example a straightened position, a bent position, a retroflected position, and/or other selected position.

In some embodiments, if the emergency button is accidently pushed, it may be released manually, for example by rotating the button counter clockwise.

FIGS. 22B-C show an example of manual release of the surgical arms using a dedicated tool. In some embodiments, tool 6005 is shaped to engage a slot 6007 configured on motor unit 6009 for opening a locking door 6011 through which the surgical arm actuators can be accessed. In an example, tool 6005 comprises a flat tip 6013 that fits within the slot to push the door from underneath. (In some embodiments, the locking door is a different door than the one through which the surgical arm is loaded to the motor unit.).

In some embodiments, surgical arm actuators such as driving cogs are exposed upon lifting of the locking door 6011. Tool 6005 can then be used for engaging the cogs (e.g. via slots) and manually rotating the cogs. Optionally, rotation of the cogs straightens the surgical arms, which can then be extracted from the body. In an example, as shown in FIG. 22C, cogs which actuate the shoulder joint (6017) the elbow joint (6019) and the wrist joint (6021) of the surgical arm are rotated.

### A Surgical Setup for Transvaginal Access

System components and/or methods as described in PCT application number IL2018/050934 and in U.S. application Ser. No. 16/109,891 (such as a mounting block, insertion and/or alignment and/or spacing handle, instrument holder, port, trocar, cannula, surgical arms, methods for access through the vagina) are incorporated herein by reference.

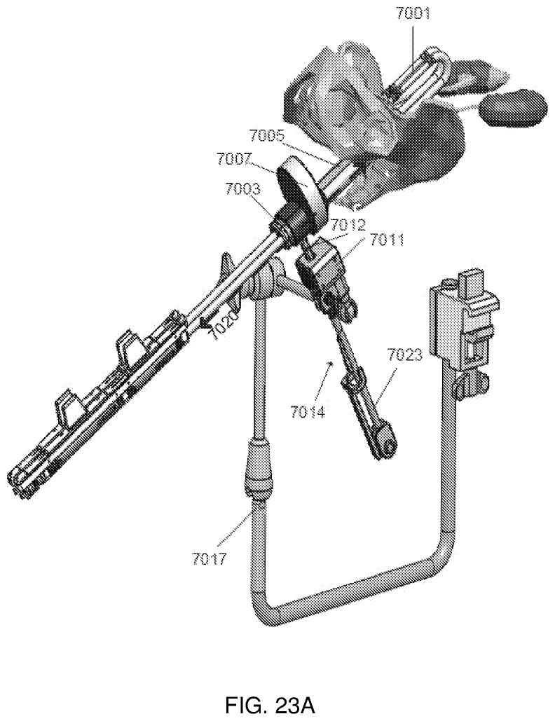

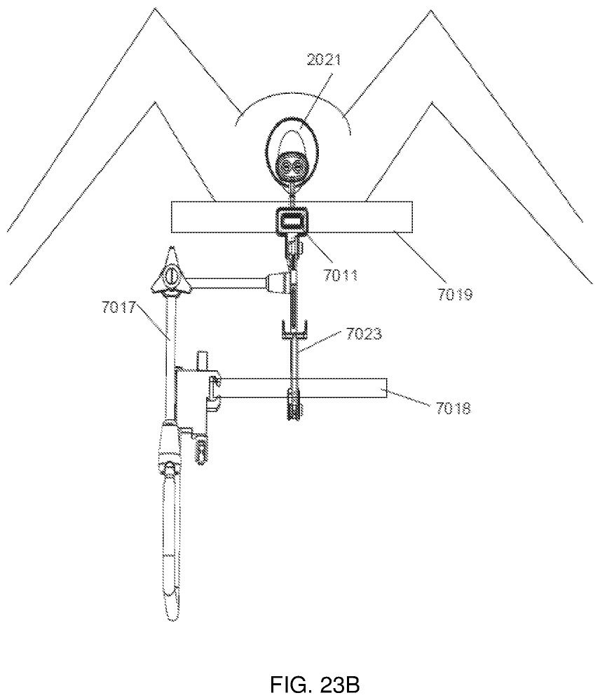

FIGS. 23A-B schematically illustrates an exemplary surgical setup for transvaginal access, according to some embodiments.

FIG. 23A shows an isometric view of the surgical setup, when the surgical arms are advanced into the patient's pelvis; FIG. 23B schematically shows a front view of the setup, according to some embodiments.

In the exemplary setup, one or more surgical arms 7001 (in this example, two arms, shown in a retroflected position) are inserted through a cannula 7003 which in turn is inserted through a trocar 7005. In some embodiments, trocar 7005 is positioned through a surgical port (for example, a gel access port 7007) located adjacent the vagina, for example in between the patient's legs, adjacent the vaginal opening.

In some embodiments, mechanical support is provided for holding the assembly of the port, trocar and cannula inserted therein. In some embodiments, a fixation arm 7017 (also referred to herein as a GYN fixation arm, for example a “FISSO” Fixation arm, sterile “FISSO” fixation arm) supports the assembly by extending from a mounting block 7011 which extends from underneath the port, for example by means of an adjustable shaft 7012. Optionally, adjustable shaft 7012 extends from trocar 7005, connecting the trocar directly to the mounting block. In some embodiments, fixation arm 7017 extends from the mounting block to an external fixed reference, such as to the surgical bed 7019 (see FIG. 23B, in this example being clamped onto a lower surface 7018 of the bed). Additionally or alternatively, the fixation arm attaches to a stable reference such as, for example, the system control console (e.g. if the surgery is performed in a small operation room).

In some embodiments, during use, the surgeon selects an entrance angle into the vagina 2021 (see FIG. 23B), for following insertion of the surgical arms. In some embodiments, the insertion angle is selected as one which avoids undesired contact of the cannula and/or surgical arms with certain tissue or organs, for example the uterus or the rectum. In some embodiments, the insertion angle is aimed towards the Pouch of Douglas.

Then, in some embodiments, the gel point port is placed next to the vagina, e.g. on an external entry to the vagina. A trocar is introduced through the port, followed by a cannula. Optionally, a distal end of the cannula extends into the Pouch of Douglas, so that when the surgical arms are advanced through the assembly the peritoneal space may be accessed via the Pouch of Douglas.

In some embodiments, the fixation arm is pre-attached to the mounting block 7011, for example via a threaded coupling. Optionally, at this point, the mounting block is attached to the trocar and cannula assembly, for example by a click-type attachment. Optionally, the click-type attachment comprises receiving the adjustable shaft 7012 which extends from the trocar, in a respective recess of the mounting block. Then, in some embodiments, an opposite end of the fixation arm 7017 is attached to a fixed reference, such as the surgical bed.

In some embodiments, independently to preparation of the above setup, the motor unit (not shown herein) for actuation of the surgical arms is attached to the motor unit fixation arm (see for example FIG. 18C, 2382), and the one or more surgical arms are loaded onto the motor unit.

Once the port and cannula are affixed in position, the user (e.g. surgeon) directs the motor unit such that the surgical arms are axially aligned with respect to the cannula, and the arms can then be advanced distally through the port.

A potential advantage of supporting the port, trocar and cannula assembly via external support such as a fixation arm that extends to the surgical bed may include reducing a load on the vaginal wall through which the trocar (and cannula) extend, thereby preventing or reducing damage such as tearing of the vaginal wall. A potential advantage of fixating the assembly at a selected location and orientation with respect to the patient's body may include preventing or reducing a risk of the assembly (or parts thereof) sliding into and/or out from the vagina. Due to the low thickness of the vaginal wall, the wall may not be durable enough to sustain and support the assembly on its own (e.g. as compared to an abdominal wall, in which a port is positionable without external support).

In some embodiments, mounting block 7011 is a component of an instrument holder 7014, which further comprises an insertion and/or alignment and/or spacing handle 7023 which extends from mounting block 7011, for guiding insertion of the surgical arms 7001.

In some embodiments, attachment of the fixation arm to at least one of the components of the assembly (trocar, port, cannula), directly or indirectly (e.g. via the mounting block), is performed in a manner in which at least an end segment of the fixation arm is positioned at an angle (e.g. 30 degree angle, 60 degree angle, 90 degree angle, 120 degree angle) with respect to a long axis 7020 of the assembly. A potential advantage of the fixation arm not being linearly aligned with axis 7020 (such as extending directly from the assembly in continuous straight line) may include reducing a footprint of the complete construct (including for example the fixation arm, port, trocar, cannula, instrument handle), interfering less with access to the surgical port positioned between the patient's legs.

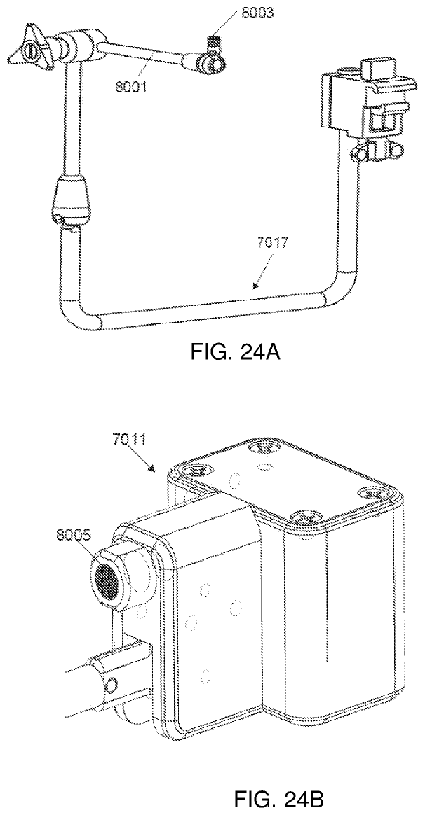

FIGS. 24A-B are drawings of a fixation arm (8A) configured to engage a mounting block (8B), according to some embodiments.

In some embodiments, fixation arm 7017 for example as described hereinabove includes an end segment 8001 which is shaped and/or sized to engage mounting block 7011. Additionally or alternatively, in some embodiments, segment 8001 is shaped to receive an adaptor which in turn is shaped and/or sized to engage mounting block 7011. In some embodiments, as shown in this example, the attachment comprises a threaded coupling, for example via a threaded screw 8003 extending from fixation arm 7017 which is received within a respective cavity 8005 formed in mounting block 7011. Optionally, cavity 8005 is internally threaded. Other coupling methods of the fixation arm to the mounting block may include a magnetic attachment, an interference coupling (e.g. a protrusion and respective recess), and/or other couplings configured to fixedly attach the fixation arm to the mounting block.

A potential advantage of coupling between the assembly and the fixation arm via the mounting block may include shifting the weight of the fixation arm onto the mounting block (and/or to other portions of the instrument holder, where the mounting block is a component of the holder), for example as opposed to the weight of the fixation arm heavying down on the assembly, thereby reducing the load on the delicate tissue. Additionally or alternatively, in some embodiments, the fixation arm is attached directly to the trocar and/or to the cannula, for example by means of a wrap around the fixation arm which grasps the arm with respect to the trocar and/or cannula.

In some embodiments, an attachment between the mounting block and the fixation arm is performed prior to the surgery, optionally during manufacturing of these tools. Optionally, during operation, a construct including the mounting block and the fixation arm is then attached to the assembly (to one or more of the port, trocar, and cannula). In an example, the mounting block is attached to an extension of the trocar.

In addition to the features of the above disclosure that are claimed in the appended claims, features believed to be inventive in their own right are set out in the clauses below, to provide fair basis for eventual filing of one or more divisional patent applications.

Clause 1. A method for accessing a treatment zone through the vagina, comprising:
selecting an entry angle;
positioning a surgical port adjacent the vaginal opening;
introducing a trocar through said surgical port;
introducing a cannula through said trocar and advancing said cannula such that said cannula enters through a posterior wall of the vagina;
externally supporting the port, trocar and cannula assembly using an extension, said extension attached on one end to at least one component of said assembly and on the other end to a fixed, stable reference; said extension holding said assembly at a fixed position in which said trocar and cannula are slanted at said selected entry angle.

Clause 2. The method according to clause 1, wherein said fixed reference comprises a surgical bed on which the patient lays.

Clause 3. The method according to clause 1, wherein said extension comprises an adjustable fixation arm.

Clause 4. The method according to clause 1, comprising, prior to said selecting, identifying a location of the Pouch of Douglas, and selecting said entry angle according to said location.

Clause 5. The method according to clause 1, wherein said holding comprises locking said assembly in position with respect to at least one of: the patient body and the surgical bed.

Clause 6. The method according to clause 1, wherein said supporting comprises reducing a weight load on said posterior wall of the vagina by shifting the weight of the assembly onto said extension.

Clause 7. The method according to clause 1, further comprising introducing one or more surgical arms through said cannula.

Clause 8. A method of controlling via one or more input device, one or more surgical mechanical arms insertable into a body of a patient, each surgical mechanical arm comprising a plurality of movable joints; the method comprising:
in a first mode of operation, navigating said surgical mechanical arms into the patient's body;
in a second mode of operation, carrying out surgical acts using said surgical mechanical arms;
wherein in said first mode of operation movement of each of said surgical mechanical arms is restricted to movement of a single joint out of said plurality of joints, and to linear movement of said surgical mechanical arm as a single unit.

Clause 9. The method according to clause 8, wherein said navigating comprises retroflecting said surgical mechanical arms within the patient's body.

Clause 10. The method according to clause 8, wherein in said first mode of operation said surgical mechanical arms are controlled by thumb operated input.

Clause 11. The method according to clause 10, wherein in said second mode of operation said surgical arms are controlled by avatar input arms.

Clause 12. The method according to clause 8, wherein said surgical mechanical arms are controlled by haptic handles during both said first and said second modes of operation.

Clause 13. The method according to clause 8 and wherein in said first mode of operation manipulation of said input device by a user is translated to a speed of movement of said surgical mechanical arm, and wherein in said second mode of operation displacement of said input device by said user is translated to a relative displacement of said surgical mechanical arm.

Clause 14. The method according to clause 8, wherein in said second mode of operation a clutch-like mode is enabled, disconnecting control of the surgical arm by the one or more input device.

Clause 15. A control console for control of one or more surgical mechanical arms according to the method of clause 8, comprising:
thumb operated input for controlling said first mode of operation;
hand operated input for controlling said second mode of operation; and
a screen interface.

Clause 16. The control console according to clause 15, wherein said thumb operated input comprises a nipple engageable by the user's thumb; whereby pushing of said nipple from a central rest position actuates respective movement of said surgical mechanical arm; and wherein a speed of said movement is affected by an extent in which the nipple was pushed relative to its rest position.

Clause 17. The control console according to clause 15, wherein in said second mode of operation manipulation of said hand operated input by a user is translated to a similar articulation of said surgical arm.

Clause 18. An imager for insertion into the body of a patient, comprising:
an elongate shaft;
a flexible extension positioned within said shaft and extendible outwardly from said shaft; and
a camera mounted onto a distal end of said flexible extension, wherein at least one of said camera and said flexible extension are positionable at a retroflected position which directs the camera backwards, opposite the direction of advancement of said elongate shaft.

Clause 19. The imager according to clause 18, wherein said elongate shaft comprises a window at a side wall of said shaft, said flexible extension configured to protrude outwardly from said window and to position said camera at an angle with respect to a longitudinal axis of said shaft.

Clause 20. The imager according to clause 19, wherein said imager is insertable into the body along with one or more surgical arms, and wherein at least said flexible extension is configured to be retroflected along with said surgical arms.

Clause 21. The imager according to clause 18, wherein said elongate shaft comprises a tissue spacer extending from a distal end of said shaft, said tissue spacer ending with a ring through which said flexible extension can pass.

Clause 22. The imager according to clause 19, further comprising a tensile element extending between a distal end of said flexible extension and said shaft, said tensile element configured to be pulled on to change a curvature of said flexible extension.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. An imaging device for capturing an image within a human body, the imaging device comprising:
a. an elongate mechanical arm; and
b. an imaging assembly coupled to the arm at a distal arm-location, the imaging assembly comprising:
i. a camera,
ii. a pivot member defining a pivot axis orthogonal to a longitudinal centerline of the elongate arm at the distal arm-location, the pivot member engaged with the camera such that the camera is pivotable about the pivot axis throughout a pivot range, and
iii. an actuation member configured to pivot the camera in response to a remote user input,
wherein the pivot range includes an orientation at which the camera is rotated at least 90° from the longitudinal centerline of the elongate arm at the distal arm-location.

2. The imaging device of claim 1, wherein pivoting the camera includes pivoting the camera to an orientation that is rotated at least 135° from an orientation of the elongate arm at the distal arm-location.

3. The imaging device of either one of claim 1 or 2, wherein the elongate mechanical arm is remotely user-manipulable.

4. The imaging device of any preceding claim, wherein the elongate mechanical arm is configured for flexing.

5. The imaging device of any preceding claim, wherein the elongate mechanical arm comprises a plurality of arm segments connected serially by arm joints having respective degrees of freedom and configured to flex and rotate in response to remote user inputs.

6. The imaging device of any preceding claim, wherein the pivot range includes at least a range of 90° at least in a plane parallel to a longitudinal centerline of the elongate arm at the distal arm-location.

7. The imaging device of any preceding claim, wherein the pivot member is transversely connected to the distal portion of the elongate arm.

8. The imaging device of any preceding claim, wherein the pivot range is independent of a distal-arm-location orientation.

9. The imaging device of any preceding claim, wherein the actuation member is powered by a local power source at the distal arm-location.

10. The imaging device of any one of claims 1 to 8, wherein the actuation member is powered by a remote power source.

11. The imaging device of any preceding claim, wherein the actuation member includes a micro-electromechanical system.

12. The imaging device of any preceding claim, wherein the remote user input is received locally at the distal arm-location by the actuation member.

13. The imaging device of any preceding claim, wherein the imaging assembly additionally includes electronic communications circuitry in data communication with the actuation member and configured to receive a remote user input.

14. The imaging device of any one of claims 1 to 11, wherein the remote user input is received remotely by the actuation member.

15. The imaging device of any one of claims 1 to 11, wherein the remote user input is received in a proximal portion of the elongated arm.

16. The imaging device of any preceding claim, wherein the pivot range includes at least a range of 90° in at least the plane parallel to the longitudinal centerline of the elongate arm at the distal arm-location.

17. The imaging device of any preceding claim, wherein the pivot range includes at least a range of 135° in at least the plane parallel to the longitudinal centerline of the elongate arm at the distal arm-location.

18. The imaging device of any preceding claim, wherein the camera is pivotable about multiple pivot axes.

19. The imaging device of claim 18, wherein the pivot range includes a range of 360° in at least one plane.

20. The imaging device of any preceding claim, wherein the pivot member is part of a 3-axis gimbal arrangement disposed at the distal-arm location.

21. The imaging device of any preceding claim, wherein the pivot range includes a range defining a pivoting of the camera about the longitudinal centerline of the elongate arm at the distal arm-location.

22. A method of capturing an image, the method comprising:
a. providing an imaging device comprising:
i. a remotely-manipulable elongate mechanical arm, and
ii. an imaging assembly coupled to the arm at a distal arm-location, the imaging assembly comprising a camera, a pivot member defining a pivot axis orthogonal to a longitudinal centerline of the elongate arm at the distal arm-location and engaged with the camera, and an actuation member configured to pivot the camera about the pivot axis;
b. receiving, by the actuation member, a remote user input;
c. in response to the remote user input, pivoting the camera, by the actuation member, in a pivot range that includes an orientation at which the camera is rotated at least 90° from the longitudinal centerline of the elongate arm at the distal arm-location; and
d. subsequent to the pivoting, capturing an image at the imaging location.

23. The method of claim 22, wherein pivoting the camera includes pivoting the camera in a pivot range that includes an orientation at which the camera is rotated at least 135° from the longitudinal centerline of the elongate arm at the distal arm-location.

24. The method of claim 23, wherein the navigating includes remotely manipulating the elongate mechanical arm.

25. The method of either one of claim 23 or 24, wherein the navigating includes flexing the elongate mechanical arm.

26. The method of claim 25, wherein the elongate mechanical arm is a remotely-manipulable arm comprising a plurality of arm segments connected serially by arm joints having respective degrees of freedom and configured to flex and rotate in response to remote user inputs.

27. The method of any one of claims 22 to 26, wherein pivoting the camera by the actuation member includes pivoting the camera in a plane parallel to the longitudinal centerline of the distal portion of the elongate arm.

28. The imaging device of any one of claims 22 to 27, wherein the pivot member is transversely connected to the distal portion of the elongate arm.

29. The method of any one of claims 22 to 28, wherein the pivot range is independent of a distal-arm-location orientation.

30. The method of any one of claims 22 to 29, wherein pivoting the camera by the actuation member includes receiving electrical power from a local power source at the distal arm-location.

31. The method of any one of claims 22 to 30, wherein pivoting the camera by the actuation member includes receiving electrical power from a remote power source.

32. The method of any one of claims 22 to 31, wherein the actuation member includes a micro-electromechanical system.

33. The method of any one of claims 22 to 32, wherein receiving the remote user input by the actuation member includes receiving the remote user input locally at the distal arm-location.

34. The method of any one of claims 22 to 33, wherein the imaging assembly additionally includes electronic communications circuitry in data communication with the actuation member, and receiving the remote user input includes receiving the remote user input by the electronic communications circuitry.

35. The method of any one of claims 22 to 34, wherein receiving the remote user input by the actuation member includes receiving the remote user input remotely.

36. The method of any one of claims 22 to 35, wherein receiving the remote user input by the actuation member includes receiving the remote user input in a proximal portion of the elongated arm.

37. The method of any one of claims 22 to 36, wherein pivoting the camera includes pivoting the camera through a pivot range of at least 90°.

38. The method of any one of claims 22 to 37 wherein pivoting the camera includes pivoting the camera through a pivot range of at least 135°.

39. The method of any one of claims 22 to 38, wherein pivoting the camera includes pivoting the camera to an orientation that is rotated at least 90° from an orientation of the elongate arm at the distal arm-location.

40. The method of any one of claims 22 to 40, wherein pivoting the camera includes pivoting the camera about multiple pivot axes.

41. The method of claim 40, wherein the pivot range includes a range of 360° in at least one plane.

42. The method of any one of claims 22 to 41, wherein pivoting the camera includes pivoting the camera in a 3-axis gimbal arrangement disposed at the distal-arm location.

43. The method of any one of claims 22 to 42, wherein pivoting the camera includes pivoting the camera about the longitudinal centerline of the elongate arm at the distal arm-location

44. An imaging device for capturing an image within a human body, the imaging device comprising:
a. an elongate mechanical arm; and
b. an imaging assembly coupled to the arm at a distal arm-location, the imaging assembly comprising:
i. a camera,
ii. a pivot member defining a pivot axis orthogonal to a longitudinal centerline of the elongate arm at the distal arm-location, the pivot member engaged with the camera such that the camera is pivotable about the pivot axis throughout a pivot range, and
iii. an actuation member configured to pivot the camera in response to a remote user input,
wherein the pivot range includes at least a range of 45°.

45. A method of capturing an image within a human body, the method comprising:
a. navigating, to an imaging location within the body, an imaging device comprising (i) an elongate arm and (ii) an imaging assembly coupled to the arm at a distal arm-location, the imaging assembly comprising a camera, a pivot member defining a pivot axis orthogonal to a longitudinal centerline of the elongate arm at the distal arm-location and engaged with the camera, and an actuation member configured to pivot the camera about the pivot axis;
b. receiving, by the actuation member, a remote user input;
c. in response to the remote user input, pivoting the camera by the actuation member, through a pivot range of at least 45°; and
d. subsequent to the pivoting, capturing an image at the imaging location.
